(19) United States
(12) Patent Application Publication
(10) Pub. No.: US 2011/0177590 A1
(43) Pub. Date: Jul. 21, 2011

(54) Bioprinted Nanoparticles and Methods of Use

(75) Inventors: Alisa Morss Clyne, Ardmore, PA (US); Kivilcim Buyukhatipoglu, Plainsboro, NJ (US); Robert Chang, Cherry Hill, NJ (US); Wei Sun, Cherry Hill, NJ (US)

(73) Assignee: Drexel University

(21) Appl. No.: 12/966,645

(22) Filed: Dec. 13, 2010

Related U.S. Application Data
(60) Provisional application No. 61/285,750, filed on Dec. 11, 2009.

(51) Int. Cl. C12N 5/071; C12M 3/04; B05D 5/12; H01F 1/00; A61L 33/00; B05C 11/00

(52) U.S. Cl. 435/325; 435/283.1; 427/128; 427/598; 427/2.24; 427/2.25; 427/2.1; 118/696

(57) ABSTRACT

The present invention provides compositions and methods that combine the initial patterning capabilities of a direct cell printing system with the active patterning capabilities of magnetically labeled cells, such as cells labeled with superparamagnetic nanoparticles. The present invention allows for the biofabrication of a complex three-dimensional tissue scaffold comprising bioactive factors and magnetically labeled cells, which can be further manipulated after initial patterning, as well as monitored over time, and repositioned as desired, within the tissue engineering construct.

### CROSS-REFERENCE TO RELATED APPLICATIONS

The present invention claims priority to U.S. Provisional Patent Application No. 61/285,750, filed Dec. 11, 2009, the entire disclosure of which is incorporated by reference herein as if set forth herein in its entirety.

### STATEMENT REGARDING FEDERALLY SUPPORTED RESEARCH OR DEVELOPMENT

This invention was made with government support under grant number CMMI-1038769 awarded by the National Science Foundation. The government has certain rights in the invention.

### BACKGROUND OF THE INVENTION

Tissue engineering is an interdisciplinary field that uses engineering and life science principles to advance our knowledge of tissue growth, which is then applied toward the development of biological tissues, such as biological tissue substitutes far use in restoring organ function (Langer and Vacanti, 1993, Science 260:920).

Most tissue engineering techniques basically consist of seeding a tissue scaffold or culture dish with cells that are grown in an incubator. The scaffold fabrication and the cell seeding are two separate processes. These techniques are very limited in their level of sophistication. The scaffolds tend to be simple structures made out of a single material, with some post-processing techniques in the slightly more complicated scaffolds. Organized, heterogeneous cellular structures are very difficult to create, and impossible to create at the complexity level of an organ using standard techniques. Seeding these kinds of scaffolds may not be enough to stimulate the cells into responding in the desired manner. A complex scaffold is required to elicit complex behavior from a cell. A new generation of tissue scaffolds is required to take the next step in tissue engineering which essentially moves away from simple scaffolds toward complex scaffolds. A cell is a very sophisticated machine with programming built into its genetic code. A complex scaffold takes advantage of this built-in programming through the incorporation of various biological factors that direct cell growth, migration, differentiation, and expression. In addition, constructs can be created that could help with the flow and transport of vital nutrients and oxygen, and the removal of waste products.

Layered manufacturing has been suggested as being well suited to the field of biology. This has resulted in much research being conducted within the field of computer-aided tissue engineering (CATE). Unfortunately, many Solid Freeform Fabrication (SFF) techniques are not biologically friendly, using techniques that cannot handle a wide range of wet materials, gels or solutions. Also, many SFF techniques utilize harsh solvents, high temperatures, high pressures, and other factors that are not conducive to biological systems. Many SFF techniques, such as stereolithography, fused deposition methods, and powder/binder-based techniques, are capable of creating tissue scaffolds, but cannot directly deposit cells or biological factors into the scaffold. This has resulted in the creation of different techniques to handle direct cell deposition.

Weiss, et al. have described a method for building bone tissue scaffolds using SFF (Reischmann et al. Electrotechnik and Informationsteclmik 2002 7/8:248-252; Weiss et al. Journal of Manufacturing Systems 1997 16(4): 239-248). This process consists of taking a CAD model of a three-dimensional structure of a bone implant, slicing the model into layers, taking laminated sheets of scaffold material, seeding the layers with cells or growth factors, and stacking them on top of each other. This process was designed for the purpose of constructing bone implants, not to provide a flexible process of creating various types of organs or biologically/chemically integrated systems and thus has several disadvantages with respect to construction of tissue engineering devices. For example, the method is limited in materials since soft, gel-like materials cannot be used as scaffold layers. This is a problem since many biological parts are soft or wet. Also, each layer of the scaffold is made with one type of sheet material. Thus, it is difficult to have two or more different materials within the same layer level. Accuracy and recalibration is an issue as well since the scaffold layers are moved from station to station. Thus, although a simple scaffold can be created by this method, a complex scaffold with controlled concentration gradients is difficult, if not impossible, to create. This is a serious disadvantage since cells are very responsive to even the slightest differences in concentration gradients.

Yan and Xiong, et al. have disclosed the concept of using layered manufacturing methods and multi-nozzle deposition extrusion and jetting processes (Xiong et al. Scripta Materialia 2002 46:771-776; Yan et al. Materials Letters 2003 57:2623-2628). Their process includes spraying and deposition of heterogeneous materials with different material properties. However, full CAD integration is not described, nor is there any description of the ability to import an assembly of multiple STL files for printing a complex, heterogeneous, three-dimensional structure. This is a vital design component when building complex parts such as biomimetic parts where MRI or CT data is incorporated into the final design, or integrating both biomimetic parts and non-biomimetic parts into a novel scaffold design.

A SFF method using a syringe-based system to dispense liquids, which is well suited for working with biological materials such as cells and hydrogels has also been described (Landers et al. Macromol Mater Eng 2000 282:17-21; Landers et al. Kunststoffe/plast Europe 2001 91(12): 21-23; Landers et al. Biomaterials 2002 23:4437-4447). The primary focus of this method is the building of scaffolds and seeding the scaffold. The deposition system used is a single nozzle device that requires cartridge swapping to change materials. This is not a very practical system for depositing multiple, heterogeneous materials such as different types of cells and growth factors all within the same scaffold layer. Further, it is difficult to take a multiple part assembly of STL files and print out a complex, biologically designed scaffold utilizing this method. Thus, there are limitations in this method with respect to the CAD integration aspect as well.

A syringe-based system for the extrusion of hybrid polymer materials embedded with glass using layered SFF manufacturing has also been described (Calvert et al. Materials Science and Engineering 1998 C6:167-174). This system also uses a single nozzle and does not incorporate CAD, thus being limited to simple designs written in Microsoft Qbasic. This system is not capable of creating heterogeneous designs within a single layer. Thus, this system is sufficient for creating basic scaffolds, but falls short of being able to create intricate scaffolds containing both biomimetic and non-biomimetic features.

A microsyringe deposition system has also been described (Vozzi et al. Materials Science and Engineering 2002 C20:43-47; Vozzi et al. Biomaterials 2003 24:2533-2540). This system utilizes a single-nozzle deposition system which has fine resolution, but is limited because of the glass capillary used for deposition. The glass capillary limits the range of viscosities that are usable due to pressure limits, and also limits the types of solutions and suspensions that can be deposited due to clogging. The device is envisaged for integration with CAD, but whether their working device could actually utilize STL files is unclear. Also, the single nozzle system makes multi-material, heterogeneous deposition difficult.

A single-nozzle, automated extrusion system that can utilize basic STL files has been described as well (Aug et al. Materials Science and Engineering 2002; C20:35-42). It is unclear whether this system can be utilized to produce multi-part, heterogeneous STL files. This single nozzle process also makes constructing complex parts very difficult, and limits the diverse range of materials available for deposition.

Mironov, et al. discuss the basic principles of organ printing, which involves direct deposition of cells using a multi-nozzle printing system (Mironov et al. TRENDS in Biotechnology 2003 21(4):157-161). A general basic concept of organ printing involving CAD in the preprocessing stage incorporating either patient specific MRI/CT data or artificial computer generated biomimetic constructs is set forth. However, there is no mention of the value of CAD beyond simply imitating biology. In addition, there are serious limitations with their disclosed multi-nozzle system which uses the same type of syringe thus limiting the types of materials that can be deposited. In order to build good 3-dimensional structures, relatively viscous solutions are required, which means high pressure. High pressure, however, may not be compatible with cells. High pressure systems handling viscous materials have the problem of not being able to deposit fine structures with fine concentration gradients. Finally, there is a flaw in the process described in this reference because they do not consider the fact that CAD programs do not have heterogeneous material capabilities. Thus, they neglect a non-trivial and difficult step by assuming that they can create a multi-material part in CAD and print it out using multiple nozzles, which is not necessarily the case.

U.S. Pat. No. 6,139,574 (Vacanti, et al. Oct. 31, 2000) discloses vascularized tissue regeneration matrices formed by SFF techniques. Use of CAD and SFF techniques for the creation of tissue scaffolds is mentioned. Further, they mention the possibility of using multiple printheads and different kinds of SFF techniques. However, there is no description of direct cell deposition. The reason for this is that the method described is not biologically friendly to cells. Thus, the described method requires depositing the scaffold material and bioactive materials first to create the scaffold, and then seeding the cells externally relying upon cell migration to populate the scaffold. Further, the inkjet printing method described by Vacanti creates problems for cellular deposition unless significant steps are taken to protect cells from shear stresses that would tear the cell apart.

U.S. Pat. No. 6,143,293 (Weiss, et al. Nov. 7, 2000) discloses assembled scaffolds for three dimensional cell culturing and tissue generation. The method used is primarily oriented towards building hard, bone-type scaffold structures and creation of soft, gel-like scaffolds using this method may be difficult. Further heterogeneous capabilities are limited to materials that can be added on top of the layer, but not within the layer itself. The method of Weiss et al. also utilizes prefabricated layers thus necessitating an assembly stage, which then requires extra steps to calibrate, align, and affix the layers. Means for affixing the layers such as barbs, or other mechanical affixing means is a disadvantage that may result in later complications due to wear, bone remodeling, or incompatibilities in material properties. The method described by Weiss et al. thus lacks versatility and flexibility.

U.S. Pat. No. 6,027,744 and U.S. Pat. No. 6,171,610 (Vacanti, et al. Feb. 22, 2000 and Vacanti, et al. Jan. 9, 2001) describe guided development and support of hydrogel-cell compositions. Methods described therein use hydrogel-cell compositions as a means of tissue scaffold construction and rely upon injecting the hydrogel-cell material into the tissue scaffold. The described method does not include layered fabrication methods or CAD. Direct deposition of cells into a scaffold while constructing the scaffold is also not mentioned.

U.S. Pat. No. 6,176,874 (Vacanti, et al. Jan. 23, 2001) discloses vascularized tissue regeneration matrices formed by SFF fabrication techniques. Again, the described method does not include layered fabrication methods or CAD nor direct deposition of cells into a scaffold while constructing the scaffold.

U.S. Pat. No. 6,454,811 (Sherwood, et al. Sep. 24, 2002) discloses composites for tissue regeneration and methods of manufacture thereof. This method primarily focuses on three-dimensional printing (3DP) for tissue engineering. Although there is mention that other methods of SFF could be used, no explicit details are provided. Further, there is no mention of CAD integration, heterogeneous materials, multi-part assemblies, and multi-nozzle printing within a CAD environment. In addition, the majority of the SFF methods described are not biologically friendly for direct cell deposition. For example, stereo-lithography, selective laser sintering, and fused deposition modeling cannot directly deposit cells due to heating and toxicity issues which will kill cells. Ballistic particle manufacturing also has problems due to shear stresses that can damage cells, which are very sensitive and require low pressure or a protective method to reduce the shear stresses experienced by the cell. The described 3DP method is also unable to directly seed cells into the interior of the part that is being constructed. This process also requires post-processing in which powder, which functions both as the part and the support material, has to be removed after finishing the printing process. Thus, while this method can be used to create porous structures, the pores are filled with powder during the printing stage. It is only after printing has been completed that the powder is removed to open up the pores. Thus, cells cannot be directly printed at specific locations inside the part. Instead, cells must migrate from the outside of the scaffold, into the interior of the scaffold. This is a serious disadvantage when trying to create reproducibility between histotypic or organ culture samples. Finer features require additional post-processing, such as salt-leaching, which again makes direct cellular deposition impossible.

U.S. Pat. No, 6,547,994 (Monkhouse, et al. Apr. 15, 2003) describes a process for rapid prototyping and manufacturing of primarily drug delivery systems with multiple gradients, primarily involving a 3DP technique. These 3DP techniques share the same shortcomings as described for U.S. Pat. No. 6,454,811.

U.S. Pat. No. 6,623,687 (Gervasi, et al. Sep. 23, 2003) describes a process for producing three-dimensional objects by constructing an interlaced lattice construct using SFF to create a functional gradient material. There is brief mention of the possibility of using this technique to create tissue engineered constructs such as veins and arteries. However, there is no evidence that such technique would work.

In many applications, tissue engineering requires precise patterning of cells and bioactive components to recreate the complex, three-dimensional architecture of native tissue. These cells and bioactive factors may then need to be repositioned during tissue growth in vitro or after implantation in vivo to achieve the desired tissue properties or they may need to be removed entirely prior to implantation for biosafety concerns. Furthermore, it is difficult to noninvasively image and track cells and bioactive factors once they are incorporated into the tissue engineered construct, much less when they are implanted in vivo. Visualization of how the tissue components move and interact is critical to improving our understanding of tissue development.

Many biofabrication techniques have been developed to incorporate living cells into functionalized scaffolds in a reproducible, three-dimensional pattern (Sun and Lal, 2002, Computer Methods and Programs in Biomedicine 67:85; Sun et al., 2005, Computer-Aided Design 37:1097). Rapid prototyping (Cohen et al., 2006, Tissue Engineering 12:1325; Wang et al., 2006, Tissue Engineering 12:83), inkjet-based cell printing (Boland et al., 2003, Anatomical Record Part a-Discoveries in Molecular Cellular and Evolutionary Biology 272A:497; Varghese et al., 2005, Journal of Thoracic and Cardiovascular Surgery 129:470; Xu et al., 2005, Biomaterials 26:93), and microcontact printing (Stevens et al., 2005, Biomaterials 26:7636; Weibel et al., 2005, Langmuir 21:6436) are among the commonly used cell deposition systems for tissue engineering applications. These biofabrication methods allow initial deposition of scaffold and cells in a pre-defined pattern. However, the methods are often expensive, time consuming, require chemically modified surfaces, or cause cell damage due to high temperatures and pressures used in the deposition process. A direct cell writing system was developed for the freeform construction of biopolymer-based three-dimensional tissue scaffolds and cell-embedded tissue constructs (Khalil et al., 2005, Rapid Prototyping Journal 11:9). The direct cell writing system uses micronozzles driven by pneumatic microvalves to deposit living cells, scaffold material, and bioactive components such as growth factors in controlled amounts with precise spatial positioning. The system requires no pre-processing, is computer controlled to rapidly produce sample replicates, and operates at room temperature and low pressure to maximize cell viability. Recently, several new approaches have been proposed to actively pattern cell constructs using external forces, including dielectrophoresis (Sebastian et al., 2007, Electrophoresis 28:3821), an optical trap (Nahmias et al., 2005, Biotechnol Bioeng 92:129), or superparamagnetic nanoparticles in a magnetic field (Ino et al., 2007, Biotechnol Bioeng. 97:1309; Frasca et al., 2009, Langmuir 25:2348).

Superparamagnetic iron oxide nanoparticles have been of primary interest for both in vivo and in vitro applications because they exhibit magnetic behavior only in the presence of a magnetic field (Gupta and Gupta, 2005, Biomaterials 26:3995; Buyukhatipoglu et al., 2009, Biofabrication 1:1-9). These nanoparticles can be conjugated with proteins or loaded inside cells, are relatively non-toxic, and can be imaged by magnetic resonance imaging (MRI) or computed tomography (CT). In vivo, superparamagnetic nanoparticles have been used to target drugs to a treatment site to increase drug efficiency and reduce systemic effects (Gupta and Curtis, 2004, J Mater Sci-Mater M. 15:493); to enhance gene delivery to target cells since nanoparticles easily cross cell membranes (Dobson, 2006, Gene Ther. 13:283; Scherer et al., 2002, Gene Ther. 9:102); and to detect vascular tissues such as tumors, since iron oxide nanoparticles appear dark on MRI images (Bonnemain, 1998, J Drug Target. 6:167; Halavaara et al., 2002, Acta Radiol. 43:180). In vitro, superparamagnetic nanoparticles have been used to create high resolution, two-dimensional cell patterns on non-functionalized surfaces (Ino et al., 2007, Biotechnol Bioeng. 97:1309; Buyukhatipoglu et al., 2009, Biofabrication 1:1-9). More recently, Frasca et al, used magnetic fields and magnetic field gradients to achieve three dimensional cell patterning (Frasca et al., 2009, Langmuir 25:2348). However the ability of this technique to create complex three-dimensional shapes is highly limited since the only method of shape control is with a magnetic field gradient from magnets placed under the scaffold material.

There is a need for improved tissue engineering techniques. The need for donors for organ transplantation is a problem within this country and with increasing life expectancy and a growing aging population, the need for, organs for transplantation is ever greater. In addition to organ transplantation, there is a need for improved methods of cytotoxicity testing for the pharmaceutical, cosmetics, and food industries. The creation of sophisticated, three-dimensional organ cultures could replace simple two-dimensional cultures that are not necessarily reliable in determining cytotoxicity. In addition, organ culture, organotypic cultures, and histotypic cultures are not easily standardizable. By having an automated manufacturing process for creating artificial organ cultures, there would be standardization, and the ability to compare experimental results between different organ cultures. In addition to the need for organs and better methods of cytotoxicity testing, there are also the developing areas of biochips, bioelectronics, biosensors, bionics, cybernetics, artificial organs, and bioactive tissue scaffolds. These devices require the integration of both biological and artificial elements. Any device that could improve this integration would be a significant advance to those fields. The present invention provides methods and systems for producing devices that can satisfy the above described needs.

### SUMMARY OF THE INVENTION

A process for manufacturing complex structure is described. The process includes the steps of designing a printable structure via a computer-operable software application, converting the designed structure into a heterogeneous material and multi-part assembly model, and printing the designed structure using a device comprising a plurality of differentiated, specialized nozzles, wherein at least one of the nozzles is specialized for the deposition of at least one material comprising a magnetic particle. In one embodiment, the structure is a tissue scaffold. In another embodiment, the material comprising a magnetic particle is a magnetically labeled cell. In another embodiment, the material comprises a magnetically labeled bioactive factor. In another embodiment, the process further comprises repositioning the magnetically labeled bioactive factor on or within the structure after initial deposit via a magnetic field after printing the magnetically labeled bioactive factor. In another embodiment, the process further comprises repositioning the material comprising a magnetic particle via a magnetic field after printing at least one material comprising a magnetic particle. In another embodiment, the magnetic particle is a superparamagnetic nanoparticle. In another embodiment, the superparamagnetic nanoparticle has a diameter between about 5-50 nm. In another embodiment, the superparamagnetic nanoparticle comprises iron oxide. In another embodiment, the process further comprises using Boolean, scaling, smoothing, or mirroring to modify the design prior to conversion into a heterogeneous material and multi-part assembly model. In another embodiment, the designing further comprises incorporating data taken from MRI, CT or other patient specific data into the designed structure. In another embodiment, the designing further comprises incorporating a biomimetic and non-biomimetic feature into the designed structure. In another embodiment, printing the material comprising a magnetic particle comprises depositing magnetically labeled cells or magnetically labeled biological factors. In another embodiment, the process further comprises improving histological accuracy, cell ratios, and spatial patterning of cells in the printed structure. In another embodiment, the structure comprises an artificial organ, an artificial vasculature or channel system, or a sample for cytotoxicity testing. In another embodiment, the structure comprises a biochip, biosensor, bionic, cybernetic, mechanoactive, or a bioactive tissue scaffold. In another embodiment, the structure is used in drug delivery.

A multi-nozzle biopolymer deposition apparatus is also described. The apparatus comprises a data processing system which processes a designed scaffold model and converts it into a layered process tool path, a motion control system driven by the layered process tool path, and a material delivery system comprising a plurality of differentiated, specialized nozzles for simultaneously depositing a plurality of biopolymers having different viscosities, thereby constructing a scaffold from the designed scaffold model, wherein at least one of the nozzles deposits at least one magnetically labeled material.

Also described is a system for generating a biocompatible structure. The system comprises designing a printable structure via a computer-operable software application, converting the designed structure into a heterogeneous material and multi-part assembly model, printing the designed structure using a device comprising a plurality of differentiated, specialized nozzles, wherein at least one of the nozzles is specialized for the deposition of at least one material comprising a magnetic particle, and applying a magnetic field to reposition the at least one material comprising the magnetic particle after its deposition. In one embodiment, the at least one material comprising the magnetic particle is repositioned prior to completion of all materials being deposited. In another embodiment, the at least one material comprising the magnetic particle is repositioned after all materials have been deposited.

### BRIEF DESCRIPTION OF THE FIGURES

For the purpose of illustrating the invention, there are depicted in the drawings certain embodiments of the invention. However, the invention is not limited to the precise arrangements and instrumentalities of the embodiments depicted in the drawings.

FIG. 1 depicts a flow diagram of a system configuration of a multi-nozzle biopolymer deposition apparatus.

FIG. 2 depicts a photograph of an exemplary multi-nozzle biopolymer deposition apparatus.

FIG. 3, comprising FIGS. 3a through 3c, depicts the creation of a multi-layered scaffold in accordance with the present invention to design a porous channel structure as a CAD model (FIG. 3a), and the use of patient specific MRI/CT data to design the required anatomical replacement scaffold model (FIG. 3b), and further use of Boolean operation to produce a porous, interconnected replacement scaffold as the finished implant (FIG. 3c).

FIG. 4, comprising FIGS. 4a through 4d, depicts the creation of a triple-layered scaffold produced in accordance with the same techniques as set forth in FIGS. 4a through 4c. FIG. 4a shows the initial CAD model, followed by creation of porous outer layer (FIG. 4b), and subsequent creation of a compact middle layer (FIG. 4c). FIG. 4d shows a cutaway view of the finished triple layer implant with porous inner layer.

FIG. 5, depicting FIGS. 5a through 5c, depicts the creation of an exemplary replacement scaffold in accordance with the present invention wherein CAD-MRI/CT and Boolean operations are combined to introduce a pre-designed structural feature into the replacement scaffold. In this example, a vascular tree was created in CAD that followed a basic pathway analogous to artery-arteriole-capillary-venule-vein (FIG. 5a). Using scaling and Boolean operations a portion of an implant was quickly “vascularized” (FIG. 5b-c). CATE was used to create channels in an implant. FIG. 5a shows the vascular tree created in CAD. As shown in FIG. 5b, the CAD design was imported and resealed as STL files in Geomagic reverse engineering software). FIG. 5c shows the scaffold structure after Boolean operation.

FIG. 6, comprising FIGS. 6a through 6d, depicts the creation of a tissue scaffold designed with built-in functional components that are non-biological in nature. In this example, a drug chamber was designed in CAD (FIG. 6a). This feature was then added to the scaffold. FIG. 6b shows the scaffold before subtraction of the chamber insert from the scaffold and FIG. 6c shows the scaffold following chamber insertion. An existing vascular tree design was also incorporated into the scaffold as shown in the cutaway of FIG. 6d, which depicts both the chamber and channels of this integrated delivery system.

FIG. 7, comprising FIGS. 7a through 7c, shows various three-dimensional hydrogel scaffolds produced with the methods and apparatus of the present invention. FIGS. 7a and 7b show a three-dimensional hydrogel scaffold comprising 10 layers of calcium alginate, extruded as a 3% (w/v) alginate filament within a cross-linking solution (FIG. 7a) and simple alginate geometrical pattern (FIG. 7b). By varying the size of the syringe nozzle, the pressures used, and the type of deposition method (extrusion), alginate filaments within the 30-40 micron range (FIG. 7e) for 3% (w/v) sodium alginate solution with a 5% (w/v) calcium chloride cross-linking solution, at 0.50 psi were produced.

FIG. 8 shows results of a cell deposition/extrusion study conducted using a hydrogel produced with the apparatus and method of the present invention. For these experiments, the hydrogel was produced from alginate hydrogel mixed with human endothelial cells at a cell concentration 750,000 cells/ml with sodium alginate: 1.5% (w/v), nozzle: EFD 200 μm at pressure: 2 psi, deposition speed: 10 mm/s, and calcium chloride: 5% (w/v).

FIG. 9 shows a hydrogel from similar experiments to those depicted in FIG. 8 wherein multi-nozzle heterogeneous deposition of different materials was used to produce the hydrogel. As shown in FIG. 9, a variety of materials were simultaneously deposited, containing different alginate solutions at concentrations in the range of 0.1%-0.4% (w/v), with the lighter gray material (indicated by A) also containing an alginate microspheres suspension and a darker gray (indicated by B) chitosan hydrogel.

FIG. 10, comprising FIGS. 10A-10E, depicts a (A) schematic of an embodiment of a solid freeform fabrication-based cell writing system, (B) a pneumatic microvalve with nozzle tip printing pattern of nanoparticles mixed in alginate, (C) printed bulk samples used for cell viability tests, (D) PAEC homogenously distributed in CaCl2 crosslinked alginate, and (E) magnetically labeled PAEC homogenously distributed in alginate.

FIG. 11 depicts the results on an example experiment demonstrating that endothelial cell viability decreased in a dose-dependent manner with magnetic nanoparticles in the alginate, but printing had no effect. (A) Cell viability for cells printed with 0, 0.1, and 1.0 mg/ml nanoparticles in 1% w/v alginate solution, assessed by Alamar blue fluorescence over time. (B) Long term cell viability for cells printed with 0 and 1.0 mg/ml nanoparticles in 1% w/v alginate solution, assessed by Alamar blue fluorescence up to 6 days after printing. (n=3, #p<0.05, *p<0.01 relative to no nanoparticle sample).

FIG. 12 depicts an example experiment demonstrating that a higher viscosity alginate scaffold decreases cell viability, however the time that the effect is observed depends on printing and nanoparticles. Cell viability for PAEC in 0.5, 1 and 2% w/v alginate and 0 or 0.1 mg/ml nanoparticles at 0 (A), 12 (B), 36 (C) and 60 (D) hours after printing. (n=3, #p<0.05; relative to 1% w/v alginate sample).

FIG. 13, comprising FIGS. 13A and 13B, depicts the results of an example experiment demonstrating that cell viability decreased for cells loaded with 0.1 and 1.0 mg/ml nanoparticles, and the decrease was accentuated by printing. PAEC viability for cells printed in 1% alginate at a dispensing pressure of (A) 5 psi and (B) 2 psi. (n=3, #p<0.05, *p<0.01 relative to no nanoparticle sample).

FIG. 14 depicts the results of an example experiment demonstrating that bioprinted nanoparticles in the alginate scaffold move towards the magnet in a manner dependent on scaffold viscosity. 1.0 mg/ml nanoparticles homogenously distributed in 1%, 2% and 3% w/v alginate (A, C, E) moved towards the NdFeB magnet for the 1% and 2%, but not 3%, alginate (B, D, F). When samples were crosslinked in CaCl2 (G, I, K), nanoparticles moved more slowly and less nanoparticle movement was observed (H, J, L). Arrows indicate magnet location. Scale bar is 1 mm.

FIG. 15 depicts the results of an example experiment demonstrating that magnetically labeled cells can be moved within the alginate scaffold using a magnet. PAEC loaded with 1 mg/ml nanoparticles homogenously distributed in 0.5 and 1% w/v alginate (A, E; higher magnification B, F) and in 0.5% alginate crosslinked with CaCl2 (I higher magnification J). Cells moved toward an NdFeB magnet placed under the culture dish (C, G, I; higher magnification D, H, L). Arrows indicate magnetically labeled cells accumulated at the magnet edge.

FIG. 16 depicts the results of an example experiment demonstrating that nanoparticles printed within a three-dimensional alginate biopolymer are visible by MicroCT. Images represent sample cross sections in the (A) translational plane, (B) coronal plane and (C) sagittal plane. The red and blue lines on the translational plane (A) show the sagittal and coronal plane cuts, respectively (B, C). The green line in the sagittal and coronal plane views represents the translational plane cut. Arrows indicate nanoparticles. Scale bar is 500 μm.

FIG. 17, comprising FIGS. 17A-17H, depicts the results of an example experiment demonstrating the formation of printed shapes of nanoparticles or magnetically labeled cells in alginate that were moved to new locations using a magnetic field. (A, B) 1% alginate with nanoparticles was printed in a specified pattern. Nanoparticles were moved to the pattern tips using a magnetic field. (C, D) Nanoparticles or (E, F) magnetically labeled cells were printed in a 600 μm thick line in a 25×25 mm 2% alginate square. The printed line pattern was moved using a magnetic field. Arrow shows the magnet location. (G, H) Nanoparticles printed in a rectangle were moved towards the magnet while maintaining the rectangular pattern.

FIG. 18, comprising FIGS. 18a and 18b, depicts (a) the experimental setup for tracking nanoparticle movement in the alginate, and (b) the magnetic field along the magnet center axis increases nearer to the magnet pole.

FIG. 19, comprising FIGS. 19a-19d, depicts the results of an example experiment demonstrating that bioprinted endothelial cell viability decreased with nanoparticles and printing pressure, but the nozzle diameter has no effect. Cell viability after bioprinting with 250 gm and 410 gm diameter nozzles, 1% alginate with 0, 0.1, and 1.0 mg ml ̂-1 magnetic nanoparticles either (a) in the alginate or (b) loaded inside cells. Cell viability after bioprinting with a 250 gm diameter nozzle, 1% alginate at 5 and 40 psi printing pressure with 1.0 mg ml̂-1 nanoparticles either (c) in the alginate or (d) inside cells (n=3, #p<0.05, *p<0.01 relative to 0 mg ml̂-1 nanoparticle sample).

FIG. 20, comprising FIGS. 20a-20c, depicts the results of an example experiment demonstrating that alginate viscosity decreased with velocity, but only high nanoparticle concentrations increased viscosity. Viscosity with 0, 1.0, and 5.0 mg ml̂-1 nanoparticles added for (a) 1% alginate, (b) 2% alginate and (c) 3% alginate (n=3, p<0.05).

FIG. 21, comprising FIGS. 21a and 21b, depicts the results of an example experiment demonstrating that nanoparticle velocity increased at low alginate concentrations and for larger nanoparticle clusters. (a) Nanoparticle velocity in 1% and 2% alginate biopolymer and (b) nanoparticle velocity as a function of agglomerated nanoparticle cluster size.

FIG. 22, comprising FIGS. 22a-22c, depicts the results of an example experiment demonstrating that printing pressure increased line width, but nanoparticles did not affect printing resolution. (a) Lines printed with and without nanoparticles using 250 and 410 gm nozzles and 2, 3.5, and 5 psi printing pressures. (b) Measured line width as a function of nozzle size and printing pressure. (d) Measured line width as a function of printing pressure, with and without nanoparticles, for the 410 gm nozzle.

FIG. 23 depicts the results of an example experiment comparing measured nanoparticle velocity with calculated velocity.

FIG. 24, comprising FIGS. 24a-24f, depicts the results of an example experiment demonstrating that the printing resolution for complex patterns was maintained with nanoparticles in the alginate. Shapes printed with alginate (a), (c), (e) and alginate with nanoparticles (b), (d), (f) were imaged using a CCD camera.

### DETAILED DESCRIPTION OF THE INVENTION

The present invention provides compositions and methods that combine the patterning capabilities of a direct cell printing system with the active patterning capabilities of magnetically labeled cells, such as cells labeled with superparamagnetic nanoparticles. The present invention allows for the biofabrication of a complex three-dimensional tissue scaffold comprising bioactive factors and magnetically labeled cells, which can be further manipulated after initial patterning, as well as monitored over time, and repositioned as desired, within the tissue engineering construct.

In one embodiment, a superparamagnetic iron oxide nanoparticle is loaded into a cell that is bioprinted in a scaffold, such as for example alginate, and printed using a multinozzle direct cell writing system, such as that described in U.S. application Ser. No. 10/540,968. On some embodiments, bioactive factors are magnetically labeled. In various embodiments, the magnetically labeled bioactive factors and magnetically labeled cells are imaged with, for example, MRI or CT, to visualize how tissues grow and develop. In other various embodiments, the magnetically labeled bioactive factors and magnetically labeled cells are moved from one location to another, for the purpose of repositioning them, or for the purpose of creating a gradient. In still other embodiments, at least a portion of the magnetically labeled bioactive factors or magnetically labeled cells are removed from the tissue, for the purpose of modifying tissue growth or development.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from I to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### Printing Systems, Scaffolds and Superparamagnetic Particles

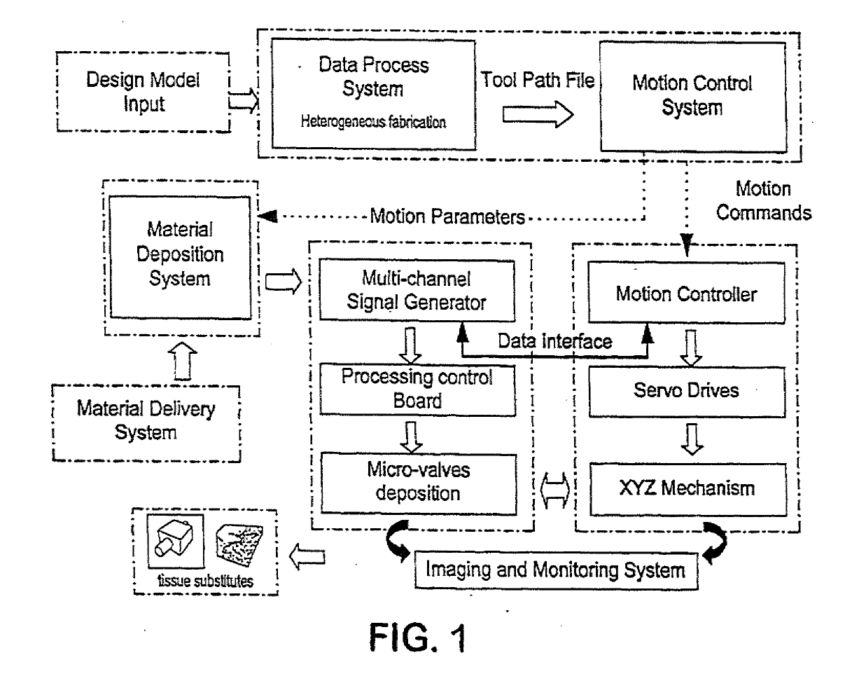

FIG. 1 depicts a flow diagram of a system configuration of a multi-nozzle biopolymer deposition apparatus of the present invention. In one embodiment, as depicted in FIG. 1, a data processing system processes a designed scaffold model and converts it into a layered process tool path. In various embodiments, the apparatus further comprises a motion control system driven by this layered manufacturing technique.

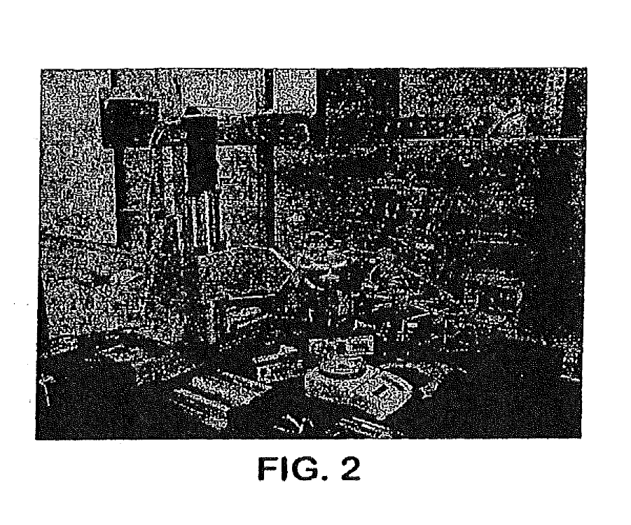

The material delivery system for the apparatus comprises multiple nozzles of different types and sizes, thus enabling the deposition of specified hydrogels having more than one viscosity for constructing a three-dimensional tissue scaffolds. In a preferred embodiment, four types of nozzles are used in the system or apparatus. In certain embodiments, at least one of the nozzles is used to deposit magnetically labeled cells. In other embodiments, at least one of the nozzles is used to deposit at least one magnetically labeled bioactive factor. Examples of nozzles include, but are not limited to, solenoid-actuated nozzles, piezoelectric glass capillary nozzles, pneumatic syringe nozzles, and spray nozzles, with size ranges varying from about 30 μm to about 500 μm. The system can continuously extrude hydrogels, or form hydrogels in single droplets with picoliter volumes. The multiple nozzle capability allows for simultaneous deposition of cells, magnetically labeled cells, growth factors, magnetically labeled growth factors, and scaffold materials, thus enabling the construction of heterogeneous scaffolds with bioactive compounds, or establishing and/or modifying functional gradient scaffolds with different mechanical/structural properties in different scaffold regions. FIG. 2 provides a photograph an exemplary multi-nozzle biopolymer deposition apparatus of the present invention.

In embodiment of the invention described herein, the apparatus and methods comprise integrated computer-aided design capabilities. In another embodiment, the invention may further comprise the use of data derived from, for example, an MRI or CT. It is an aspect of the apparatus and methods of the invention that it can utilize patient specific data, thereby allowing adaptation of each manufactured part of a person's unique geometry.

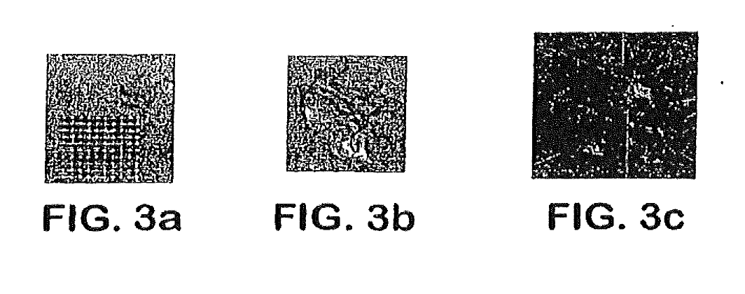

CAD provides a user with the basic ability to create both biomimetic and non-biomimetic designs and features. These can be created by the deposition of electrically conductive materials, magnetic materials, thermally conductive materials, mechanically active materials, bioactive elements, genetic materials and vectors, and so forth. For example, as shown in FIG. 3a through 3c, CAD can be used to design a porous channel structure as a CAD model (FIG. 3a). Patient specific MRI and/or CT data can be used to design the required anatomical replacement scaffold model (FIG. 3b). Boolean operation is then used to produce a porous, interconnected replacement scaffold (FIG. 3c).

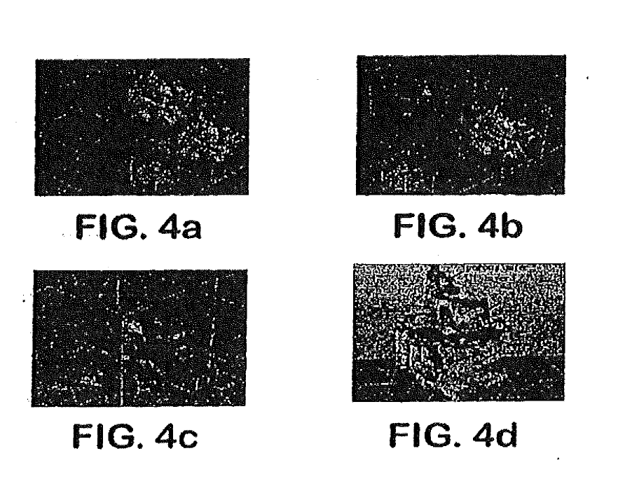

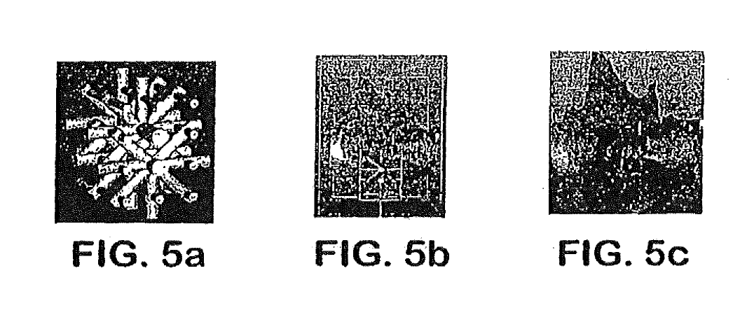

As shown in FIG. 4a through 4d, using these same methodologies, a triple-layered structure with a porous outer layer, a compact middle layer, and a porous inner layer is created. Further, as shown in FIG. 5a through 5c, CAD-MRI/CT and Boolean operations can be combined in accordance with the present invention to introduce a pre-designed structure feature into a scaffold such as replacement scaffold. For example, as shown in FIG. 5, a vascular tree can be created in CAD that follows a basic pathway analogous to artery-arteriole-capillary-venule-vein (FIG. 5a). Using scaling and Boolean operations a portion of an implant can then be quickly “vascularized.”

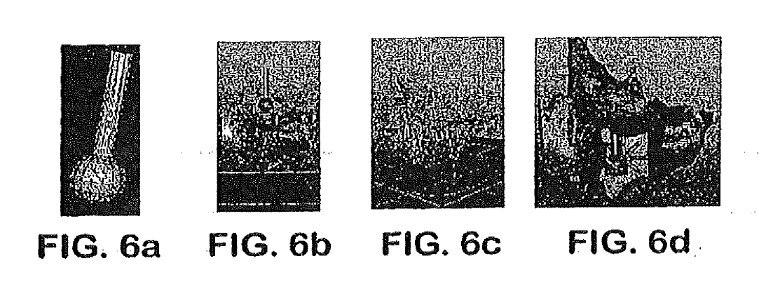

With the power of the computer-aided tissue engineering, tissue scaffolds can be designed with built-in functional components that are non-biological in nature. For example, growth factors and drugs play vital roles in tissue engineering. Accordingly, a drug chamber for storage and delivery of such agents can be designed in CAD (FIG. 6a) and then added as a feature to the scaffold (FIG. 6b-d). As shown in FIG. 6d, an existing vascular tree design can also incorporated into the scaffold. Other non-biomimetic features such as inlet and outlet ports and attachment interfaces can be added in similar fashion thus allowing for quick assembly of sophisticated scaffolds using the methods and apparatus of the present invention.

As shown by these exemplary scaffold embodiments of FIGS. 3 through 6, following CAD design, the methods and the apparatus of the present invention may further comprise Boolean, scaling, smoothing, mirroring, and/or other modifying operations which can be used to design and incorporate biomimetic and non-biomimetic features. Thus, various embodiments of the method of the present invention may comprise the use of Boolean, scaling, smoothing, mirroring, and/or other operations to modify the design. A combination of these types of operations adds great versatility to the design process. Examples of Boolean operations are addition and subtraction operations used to create voids or parts that fill voids, conforming to their geometry and anatomical shape. Boolean additive and subtractive capabilities also allows the operator to create a set of standardized or “stock” parts and features that can be reused and recycled in multiple designs. While such operations can be skipped when creating relatively simple devices, when building complex devices, use of one or more of these operations are extremely useful and expand the design capabilities immensely.

The ability to create both biomimetic and non-biomimetic features permits one of skill in the art to produce a device such as a scaffold comprising, for example, electrically conductive materials, magnetic materials, thermally conductive materials and mechanically active materials as well as bioactive elements, genetic materials and vectors. Examples of non-biomimetic features which can be incorporated into devices produced by this method and apparatus include, but are not limited to, electrically conductive material deposited, extruded, laid down, in order to create wires, circuits, biochips, etc., mechanically active elements such as microvalves or miniature pumps and actuators built or incorporated into the finished part to create a microfluidic device, biochip, biosensor, a specialized component or prefabricated element, such as an integrated circuit, valve, or piezoelectric element added through an automated device that is designed to place it into the part being constructed, a tip or other device used to direct electrical stimulation or to apply a charge to direct ion flow, stimulate muscle contraction, cause changes to the cell nucleus, and a tip or device with a voltage potential between the tip and substrate in order to deposit material onto the substrate via a process similar to electrospinning.

In some embodiments, the methods and apparatus of the present invention further comprise multi-nozzle capability thus permitting the deposit of multiple materials, including magnetically labeled cells and/or growth factors, within the same layer. Different types of specialized nozzles provide versatility to the process of the present invention to handle a wide range of materials such as cells, magnetically labeled cells, bioactive factors, magnetically labeled bioactive factors, suspensions, gels, and a wide range of viscosities ranging from that of water to that of viscous glues. Further, multiple modes of nozzle operation can be provided including, but not limited to, droplet deposition, extrusion, and spraying operations, thereby allowing control of different levels of resolution and material properties. For example, fine microdroplet deposition may be used for adding minute concentrations of biological factors, and extrusion may be used to create a strong scaffold structure.

Accordingly, in various embodiments of the method and apparatus of the present invention, interface software is used to convert the CAD designed device of step 1 or steps 1 and 2 into a heterogeneous material and multi-part assembly model that can be used for multi-nozzle printing. This is an important step of the method as it allows the user to take a multi-material CAD design and print it out using multiple nozzles.

The methods and apparatus of the present invention may further comprise heterogeneous material and multi-part assembly capabilities so that in the methods of the present invention the design is printed out using the different, specialized nozzles. This aspect also vastly increases the repertoire of materials that can be utilized, and thus expands the type of designs that can be built, ranging for example from biological to non-biological scaffolds, parts, devices, etc. The nozzles are also capable of handling multiple modes of nozzle operation such as droplet deposition, extrusion, and spraying, thus allowing for control of different levels of resolution and material properties. An aspect of the present invention is that the combination of the initial patterning capabilities of a direct cell printing system with the ability to actively pattern magnetically labeled cells at times following initial patterning provides for modifications to be made to the positions of magnetically labeled cells. By way of a non-limiting example, modifications to the position of magnetically labeled cells can be made to reposition cells due to suboptimal migration. It is also an aspect of the present invention is that the combination of the initial patterning capabilities of a direct cell printing system with the ability to actively pattern magnetically labeled bioactive factors at times following initial patterning provides for modifications to be made to the positions of magnetically labeled bioactive factors, thereby affecting the development of the growing tissue.

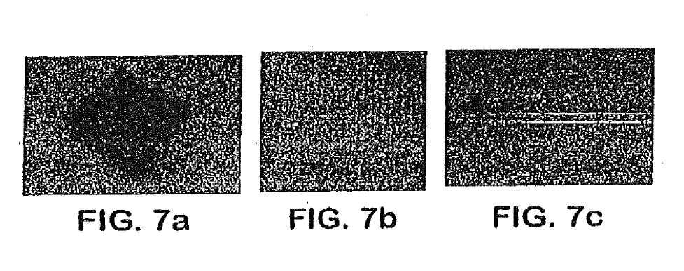

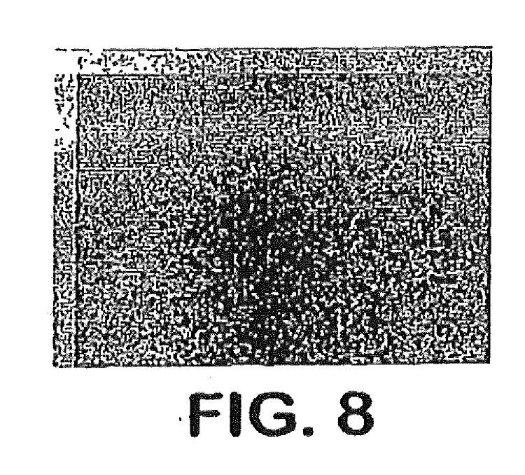

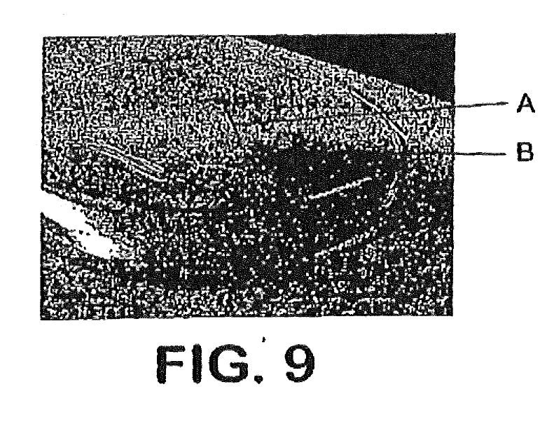

Exemplary hydrogels depicting versatility achieved through use of the apparatus and methods of the present invention are depicted in FIGS. 7 through 9. As can be seen, using the method and system of the present invention a complex, multi-material CAD design can be printed out using multiple nozzles. This is a significant advantage of the methods and system of the present invention that cannot be accomplished using-CAD and solid modeling programs incapable of modeling heterogeneous parts with different material properties.

The methods and apparatus of the present invention utilize biologically friendly design capabilities so that cells, magnetically labeled cells, biological factors and/or magnetically labeled biological factors can be deposited directly within and/or onto the scaffold.

Direct cell deposition, with the capability of modifying the position of cells after deposition, is a very important capability that is often overlooked, and is a significant difference from prior methods. Many have not considered and have failed to see its importance in creating organ cultures with reproducible samples. Being able to create organ, organotypic, or histotypic cultures by using the exact same assembly procedures with reliability will revolutionize the pharmacological, food, and cosmetics testing industries. Organ cultures will be a much more reliable indicator of true drug behavior in vivo than current cytotoxicity testing methods. This will reduce greatly the cost of drug testing and manufacturing and serve to lower the cost of medication and health care costs. It will also reduce the amount of animal testing that is done as well. Organ cultures that can be compared with each other can provide insight into other fields as well such as molecular and cell biology, genetics, and tissue engineering.

In addition, direct cell deposition, with the capability of modifying the position of cells after deposition, creates tissue structures that are more histologically accurate. That is, cells are placed next to other cells that they are normally next to within an in vivo environment. They can also be deposited in their proper location and ratios, and when magnetically labeled, can be imaged and repositioned over time. In embodiments where the cells are magnetically labeled, the positions of the magnetically labeled cells can be monitored and modified over time to correct for suboptimal positioning due to, for example, migration. As the skilled artisan will understand, cells do not exist in vivo alone, but rather rely upon each other for proper function and maintenance. Cell-cell signaling and communication either from direct contact or paracrine signaling is vital for proper cellular behavior, differentiation, and proliferation. Also, the extracellular matrix produced by cells is important for optimal cellular function.

CAD integration capabilities of the methods and apparatus of the present invention also allow for the incorporation of non-biological elements into the design including, but not limited to, drug chambers, access ports, biotelemetry for doctors and biosensors. These non-biomimetic features can be created in CAD, as shown for example in FIG. 6, saved as a part, and then reused over and over, being incorporated into many different designs. Thus, integration of CAD in the process of the present invention enables not just the building of devices that imitate nature, but also the building of devices that can assist or go beyond nature.

The multi-nozzle system with different types of nozzles used in the methods and system of the present invention permits layering of multiple components into the device. Nozzles could be different in sizes, diameters, tip types, or in different operational mechanisms, such as solenoid, piezoelectric, and pneumatic air-regulated nozzles. By way of one non-limiting example, one nozzle can be specialized for cell deposition, while another nozzle can be optimized for depositing a viscous structural member.

As shown in FIG. 1, implementation of an embodiment of the method and apparatus of system of the present invention involves the use of an automatic control system, including a computer with software for CAD and medical imaging process ability to perform Boolean operations, mirroring, smoothing and three-dimensional reconstruction from MRI/CT to tissue replacement model; a XYZ positioning system inclusive of motion controllers and motors with an XYZ axis; a multi-nozzle system preferably comprising at least a microdroplet/fine resolution nozzle and a high viscosity/extrusion nozzle, as well as nozzle controllers, fluid reservoirs, and filters; and a pressure system inclusive of pressure tanks, pressure chambers, compressor/vacuum pumps, pressure sensors, and regulators.

In addition to the above-preferred embodiment, alternative variations of the methods and system are included in the scope of the invention described herein. In one embodiment, a device can be constructed by either moving the platform that the device is being constructed on, or by moving the print head, or by a combination of both through controlling the XYZ positioning system.

Alternative nozzles or other devices can also be used to provide various coatings or washings. For example, biochemical surface treatment can be performed via a nozzle or other device, for example, by washing, spraying, etc., simultaneously with the deposition of scaffolding materials through other nozzle(s). A coating material can also be sprayed on the device simultaneously with the deposition of the scaffolding material through other nozzle(s), or a coating material can be sprayed onto a single layer or layers of the device. One of the nozzles or other device can also be used to add a support material or temporary scaffolding that can later be removed from the finished part, for example, a reversible gel, simultaneously with the deposition of the scaffolding material through other nozzle(s). One of the nozzles can also be used to deposit drops-on-demand drugs, or lines of powder or solid materials, simultaneously with the deposition of the scaffolding material through other nozzle(s). One of the nozzles can also be used to deliver energy to speed the scaffold solidification, for example, to transmit a WV or Laser through an optical fiber simultaneously with the deposition of the scaffolding material through other nozzle(s). One of the nozzles can also be used to deposit, extrude or pattern electrically conductive materials within the scaffold simultaneously with the deposition of the scaffolding material through other nozzle(s) to generate wired, circuited, or biochip embedded scaffolds. One of the nozzles can also be used to deposit cells, such as magnetically labeled cells. One of the nozzles can also be used to transmit/deposit fluid simultaneously with the deposition of the scaffolding material through other nozzle(s). The fluid can be applied to the part for various purposes such as cooling, sterilization, cross-linking, solidification, etc. When using fluids, the part can be created in a container capable of holding fluids (a dish, a culture plate well, a fluid tank, etc.). The fluid level can be incremented by the same height as the layer being formed, thus raising the fluid level, or, the height level of the part could be decremented, thus lowering it into the fluid.

In-situ sterilization can be incorporated into the method of the present invention as well and can be done in several ways. In one embodiment, a solution with antibiotics such as penicillin is added through the multi-nozzle deposition system while making the device or afterwards. In another embodiment, a sterilizing solution (non-antibiotic) is added to one of the nozzles for deposition or post-sterilization. An alternative device to a nozzle, as part of the multi-nozzle deposition system, can also be used such as device emitting ultraviolet radiation, heat, or gamma irradiation.

The methods and apparatus of the present invention can further comprise imaging capabilities such as an ultrasonic transducer that can be used for imaging the device while it is being built. Alternatively, an optical imaging apparatus, such as a microscope, can be used to provide visual information, or provide data for feedback in a closed-loop control system. An optical imaging apparatus can also be used to monitor fluorescence and reporter gene activities which can be used for cell counting, calculating the presence of proteins, DNA expression, metabolic activity, cell migration, etc. Atomic force microscopy and scanning tunneling microscopy, can also provide information about the device at nanoscale resolution.

Sensing devices can also be incorporated into the methods and system to provide relevant data such as temperature, or to monitor chemical reactions, chemicals released during production, and/or mechanical forces such as shear during production. Such sensing devices can be used to create a feedback control mechanism to regulate the process parameters in an automated fashion.

Mechanical agitation or stimulation devices such as ultrasonic, subsonic, and/or sonic transducers can also be incorporated into the methods and system to stimulate the device mechanically during construction. The stimulations will help to improve the device structural properties, for example, homogeneity of the cell and scaffolding material distribution. Further, mechanical devices can be used to stamp, press, adjust, move, cut, and trim the device during construction.

Thus, the methods and apparatus of the present invention comprise multiple steps and elements that, when combined, create a very powerful and robust method and system for manufacturing devices within the biological field, as well as other fields outside of biology. For example, devices produced in accordance with the methods and system described can be used as reproducible organ cultures. Such organ cultures are expected to be very useful in cytotoxicity testing (i.e. food, drug, and cosmetics industry), and other fields such as the study of tissue engineering, molecular biology, and cell biology. The greatly improved reproducibility between samples of organ cultures is achieved using the method and system of the present invention by directly depositing cells, such as magnetically labeled cells, and biological factors, such as magnetically labeled biological factors, while building the device. If one relies solely upon inward cell migration into the completed tissue scaffold or construct, there is less consistency in the location, distribution, or ratios of the cells, especially over time. With the automated methods and system of the present invention, reproducibility between heterogeneous, 3-dimensional organ cultures is achieved. The cells, biological factors, and scaffold materials can be precisely deposited in the same locations, in the same manner, and with the same concentrations. This results in organ cultures that are assembled in the exact same manner, and so can be used to make comparisons between different organ culture test samples. With magnetically labeled cells, cells can be monitored and re-positioned at times after initial deposition. In addition, direct cell deposition and magnetically labeled cell repositioning, using the methods and system of the present invention permits creation of tissue engineering devices that are, and remain, more histologically natural. Cells can be placed next to other cells in a spatial pattern and orientation similar to their in vivo environment. They can also be deposited in their proper ratios, thus resulting in a much better tissue scaffold than that produced by current methods. When magnetically labeled, the cells can be repositioned at times following initial patterning until the organ culture matures and physiologic cell-cell signaling and extracellular matrix produced by cells is established.

Additional ramifications of the methods and system of the present invention include scaling up and mass production. The introduction of computer-aided design and automated assembly allows for mass production of tissue samples, cultures, and organs that can be used for pharmacological testing, for example, in testing hundreds of variations of cancer-fighting drugs. Automation can lead to not only increased design complexity, but also increased speed, consistency, and quality control.

In some embodiments, magnetic nanoparticles conjugated to bioactive factors such as growth factors, antibodies, drugs, or genes can be deposited into the printed organ or tissue. In this way, bioactive factors can be precisely positioned within the three-dimensional organ or tissue scaffold. The ability to move these magnetic nanoparticles inside the scaffold makes it possible to move the bioactive factors during tissue maturation and growth, for example to provide endothelial cells with a changing growth factor gradient to promote angiogenesis. In certain embodiments, the nanoparticles can be removed prior to organ/tissue implantation, thereby decreasing the possibility of any potential negative effects in vivo.

Nanoparticle manufacturing parameters, such as those relating to size and composition, affect printed cell viability. As contemplated herein, small, polymer-coated nanoparticles can decrease bioprinted cell toxicity.

Also contemplated herein is how nanoparticle location relative to the cell affects bioprinting-induced cell damage. For example, nanoparticles can be mixed into the scaffold, loaded inside cells, or attached to the cell surface prior to printing. While cell magnetic labeling is maximal when nanoparticles are loaded inside cells, cell viability can be improved when nanoparticles are attached to the cell surface.

As demonstrated herein, nanoparticles may interact with micron-scale cells in nanomanufacturing processes, and this interaction translates into biological outcomes. As presented herein, nanoparticles can be manufactured and used in ways that minimize cytotoxicity and maintain cell function. Thus, the dynamic manipulation and tracking of cells and bioactive factors within these structures can transform tissue engineering. Because nanoparticles interact with mechanosensitive tissues, such as bone, lung and vasculature, for example, their use in imaging, cancer treatment, or even when inhaled through the environment can be significantly enhanced by the present invention, and may play a critical role in advancements in nanomanufacturing for medicine.

A fundamental understanding of nano-bio interactions is critical to a well-designed manufacturing process. The present invention can be applied across varied biological applications in which nanoparticles interact with cells under mechanical stimulation, such as from imaging and cancer treatment to drug delivery, for example. Similarly, the present invention can be applied to other nanomanufacturing applications in which nanoparticles are incorporated into devices with micron-scale features. If critical nanoparticle manufacturing parameters can be modified to maximize cell viability and function, cells and bioactive factors can be dynamically moved and imaged within 3D scaffolds. Thus, the present invention can dramatically advance tissue engineering capabilities and 3D tissue development,

In biomedical applications, iron oxide (FO₃O₄) nanoparticles are of primary interest for in viva and in vitro applications because they are superparamagnetic and improve imaging contrast. It should be appreciated that any superparamagnetic nanoparticle may be used with the present invention, as would be understood by those skilled in the art. As contemplated herein, superparamagnetic nanoparticles can be used to target drug or enhance gene delivery by immobilizing the therapeutic agent to the nanoparticle surface and guiding it to the desired cell or tissue under an applied external magnetic field. While iron oxide nanoparticles show great potential, nanoparticles can also damage cells through reactive oxygen species (ROS) formation and actin cytoskeleton disruption. These effects may alter cell function and cell response to mechanical forces.

In another aspect of the present invention, the superparamagnetic particle may vary in size. For example, in one embodiment, the superparamagnetic particles may range in diameter from about 1-100 nm, and any whole or partial increments therebetween. In other embodiments, the particles may range in diameter from about 5-50 nm, and any whole or partial increments therebetween. In still other embodiments, the particle size may be about 2 nm, about 5 nm, about 10 nm, about 15 nm, about 20 nm, about 25 nm, and about 30 nm in diameter. It is expected that smaller nanoparticles may have less damaging cell interactions during the printing process, while being taken up more readily by cells.

In another aspect of the present invention, the superparamagnetic particles can be coated with a polymer, or they can optionally not be coated with a polymer. In one embodiment, the particle can be coated with PEG. It should be further appreciated that the particles can be coated with any sort of polymer, such as polysaccharide dextran, and at any concentration, as would be understood by those skilled in the art. While it is possible that PEG or other polymer coatings may diminish nanoparticle effects on cell function through decreased intracellular ROS formation, a PEG coating can be selected due to its wide use in nanoparticle drug delivery, the capability of further modifying PEGylated nanoparticles (such as with an antibody to a specific cell surface marker such as VCAM for endothelial cells), and the relatively simple coating process. The methods provided herein can improve the potential of magnetically functionalized tissue engineering scaffolds for moving and tracking cells. Further, if endothelial cells do not express adequate VCAM to be magnetically labeled using these antibodies, selectins can also be used.

Thus, the present invention also includes a system for generating a biocompatible structures and manipulating the structure either during or after the material deposition process. For example, the system comprises designing a printable structure via a computer-operable software application, converting the designed structure into a heterogeneous material and multi-part assembly model, printing the designed structure using a device comprising a plurality of differentiated, specialized nozzles, wherein at least one of the nozzles is specialized for the deposition of at least one material comprising a magnetic particle, and applying a magnetic field to reposition at least one material comprising the magnetic particle after its deposition. In one embodiment, at least one material comprising the magnetic particle is repositioned prior to completion of all materials being deposited. In another embodiment, at least one material comprising the magnetic particle is repositioned after all materials have been deposited. It should be appreciated that the magnetic particles can be incorporated into any portion of the designed structure. For example, the magnetic particle can form part of a tissue scaffold, placed on a tissue scaffold, or be incorporated into a bioactive factor or cell that is seeded into or onto the scaffold. In alternative embodiments, the magnetic particle can be manipulated before or at any point during its deposition or incorporation into the resulting printed structure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The apparatus depicted in FIG. 2 was used to construct various three-dimensional biopolymer based tissue scaffolds. For example, shown in FIG. 7 are, several three-dimensional hydrogel scaffolds (10 layers, calcium alginate), extruded as a 3% (w/v) alginate filament within a cross-linking solution (FIG. 3a) and simple alginate geometrical pattern (FIG. 3b). Depending upon the size of the syringe nozzle, the pressures used, and the type of deposition method (extrusion), alginate filaments within the 30-40 micron range (FIG. 3c) were created for 3% (w/v) sodium alginate solution with a 5% (w/v) calcium chloride cross-linking solution, at 0.50 psi.

Further, cell deposition/extrusion studies were conducted by extruding alginate hydrogel mixed with human endothelial cells at a cell concentration: 750,000 cells/ml with sodium alginate: 1.5% (w/v), nozzle: EFD 200 μm at pressure: 2 psi, deposition speed: 10 mm/s, and calcium chloride: 5% (w/v) (see FIG. 4). Experiments were also performed testing multi-nozzle heterogeneous deposition of different materials. As shown in FIG. 5, a variety of materials were simultaneously deposited, containing different alginate solutions at concentrations in the range of 0.1%-0.4% (w/v), with the light gray material designated by A also containing an alginate microspheres suspension and a darker gray chitosan hydrogel designated as B.

### Example 1

### Nanoparticle Uptake and Cell Viability

The following materials and methods were used in Example 1.

### Chemical Formulation

Sodium alginate powder (FMCBioPolymer, Drammen, Norway) was dissolved in deionized water at 0.5, 1, 2 and 3% w/v concentrations. An ionic cross-linking solution was prepared by dissolving calcium chloride, CaCl2 (BDH Chemicals, Poole, UK), in deionized water. NanoArc magnetic iron oxide nanoparticles (Alfa Aesar, Ward Hill, Mass.) of 20-40 nm in diameter were used in all experiments. Sodium alginate-magnetic nanoparticle solutions were prepared by vigorously mixing sodium alginate with increasing concentrations of iron oxide nanoparticles to achieve a homogeneous nanoparticle distribution.

### Cell Culture

Porcine aortic endothelial cells (PAEC) were isolated by the collagenase dispersion method and maintained in low glucose Dulbecco's Modified Eagle's medium (DMEM) supplemented with 5% fetal bovine serum, 1% penicillin-streptomycin, and 2% glutamine (Invitrogen). Culture media was changed every 48 hours, and cells between passages 4 and 9 were used. Prior to printing, cells were gently mixed at a concentration of 1.5×10̂5 cells/ml in sodium alginate solution to ensure uniform cell distribution. For magnetically labeled cells, PAEC in 100 nun tissue culture dishes were loaded with different nanoparticle concentrations and incubated at 37° C. in a 5% CO₂ incubator for 24 hours.

### Cell Dispensing System

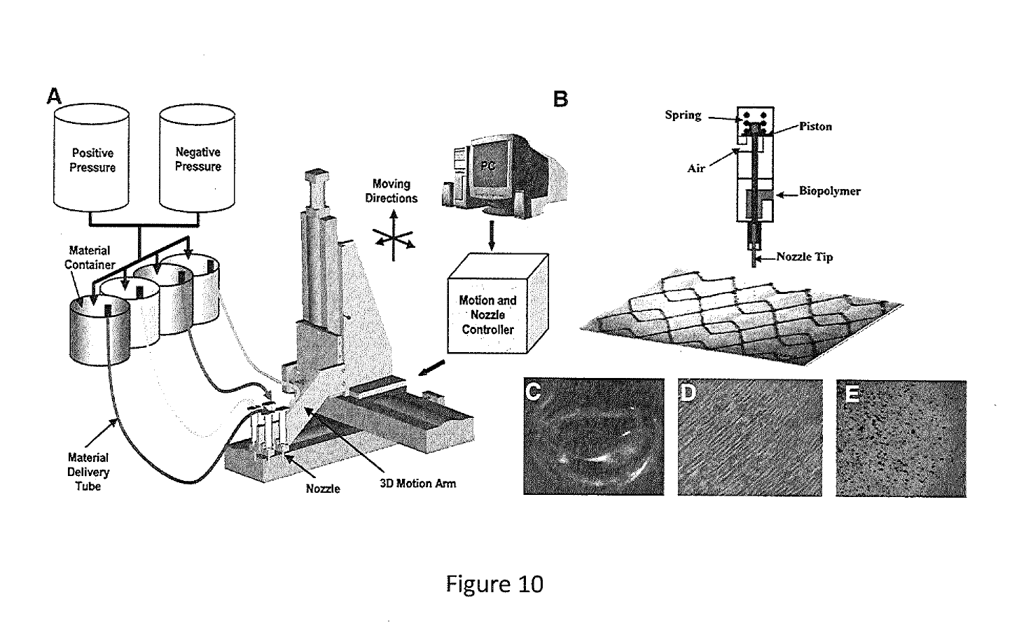

A proprietary solid freeform fabrication-based direct cell writing system (FIG. 10) was developed to create three-dimensional tissue constructs by dispensing cells and biopolymers into predefined patterns (Khalil et al., 2005, Rapid Prototyping Journal 11:9; Chang and Sun, 2008, Tissue Engineering Part A 14:41). The direct cell writing system used in this study operates at room temperature and low-pressure conditions to facilitate deposition of living cells, growth factors, or other bioactive compounds in controlled amounts with precise spatial positioning. Pneumatic microvalves (EFD, East Providence, R.I.) were used to apply a low printing pressure of 5 psi to minimize cell death from the dispensing process (Khalil et al., 2005, Rapid Prototyping Journal 11:9; Chang and Sun, 2008, Tissue Engineering Part A 14:41).

Sodium alginate was chosen as the scaffold biopolymer. Alginate-nanoparticle-cell mixtures with 0, 0.1, or 1.0 mg/ml nanoparticle concentration were printed with 250 μm nozzles. Control samples were dispensed in the system but without using nozzle tips. All samples were dispensed as 0.3 g of bulk material with a sample size of three, and each experiment was repeated a minimum of two times. Data presented are from one representative experiment. After dispensing, each sample was immediately submerged in a 5.0% w/v CaCl₂ cross-linking solution for 5 minutes, placed in supplemented media, and returned to the incubator. Samples in the long-term study were cross-linked daily to maintain both cell immobilization and alginate structural integrity. Representative images of printed bulk samples and cell distribution in alginate bulk samples are presented in FIGS. 10C, 10D and 10E.

### Alamar Blue Cell Viability Assay

Alamar blue quantitatively measures cell metabolic activity using an oxidation-reduction (REDOX) indicator that fluoresces and changes color in metabolically active cells (Nakayama et al., 1997, J Immunol Methods 204:205). Cross-linked alginate-cell solutions in 6 well plates were incubated with 2 ml supplemented media and 200 μl Alamar blue solution (AbD Serotec Ltd, Oxford, UK). After 4 hours of incubation at 37° C. in 5% CO₂ atmosphere, 100 μl of media from each well was transferred into a 96 well flat-bottomed black assay plate, and fluorescence was measured at 535/590 nm in a GENios microplate reader. 3×10̂4 cells were calibrated to a fluorescence intensity reading of 35000. Since the Alamar blue assay measures the mean metabolic activity of the cell population, cell viability was confirmed using a Live/Dead assay (Invitrogen, Carlsbad, Calif.) as per manufacturer instructions.

### Nanoparticle and Magnetically Labeled Cell Movement in the Scaffold

Bulk samples consisting of 1.0 mg/ml magnetic nanoparticles in 0.5, 1.0 and 2% w/v alginate were printed using the direct cell writing system. A 1 inch diameter NdFeB magnet with a surface field of 6450 Gauss (K&J Magnetics, Jamison, Pa.) was placed under the 60 mm cell culture dishes. Movement of magnetic nanoparticles and the magnetically labeled cells by the applied magnetic field was imaged using a 4 Megapixel CCD camera (Alpha Innotech, San Leandro, Calif.).

### Micro Computed Tomography Scan

A 1.5 mm×1.5 min area of 0.1 mg/ml magnetic nanoparticles was printed within a 5 mm×5 mm×2 mm 2% w/v alginate construct and imaged using a MicroCT scanner (SkyScan 1172). MicroCT allows non-destructive evaluation of the internal structure and composition of the sample based on changes in X-ray absorption. Image resolution was set at 2.16 μm with a filter of 1 mm aluminum. The rotation angle was 180° with a rotation step of 0.1°.

### Statistical Analysis

Samples were statistically compared using Student's t-test. Statistical significance was established at either p<0.05 (#) or p<0.01 (*). Two-way ANOVA was used to compare changes over time, with statistical significance established at p<0.0001.

The following results are provided for Example 1.

Viability of Cells Printed with Magnetic Nanoparticles in the Alginate

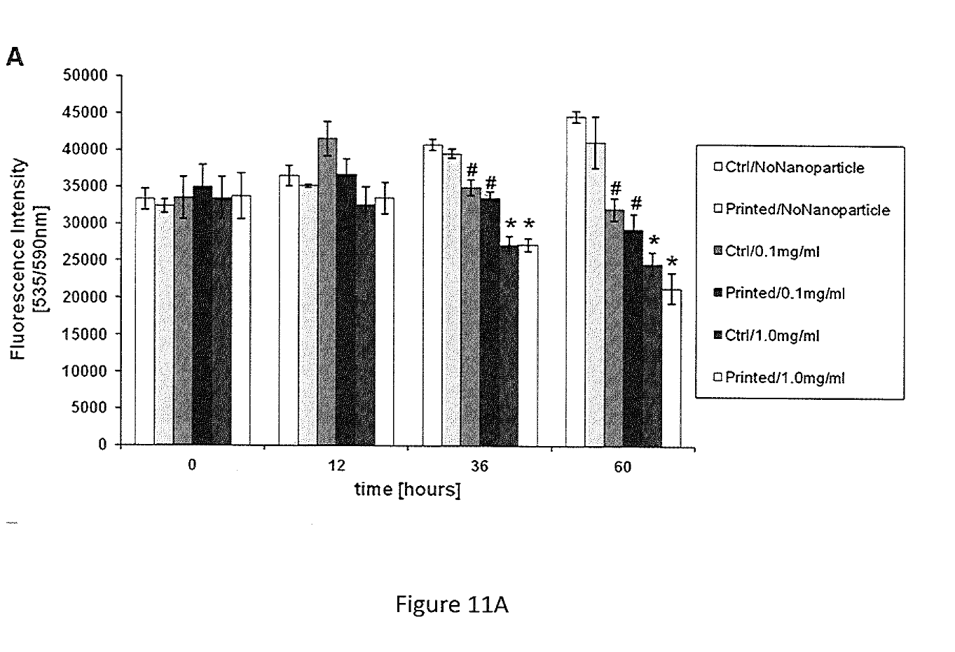

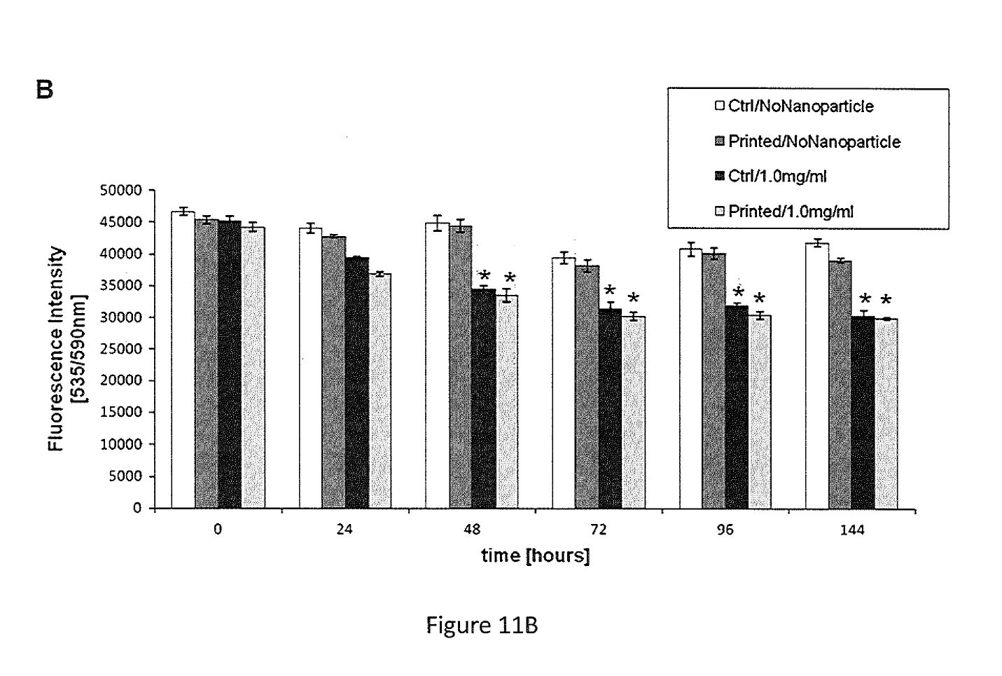

Bioprinting magnetic nanoparticles along with cells in a biopolymer scaffold may provide an effective means to track and manipulate bioactive factors in tissue engineered structures. While nanoparticles themselves slightly decreased endothelial cell viability, bioprinting had no significant effect (FIG. 11A). At 0 and 12 hours after printing, cell viability did not change significantly for unprinted or printed cells with 0 or 0.1 mg/ml nanoparticles in a 1% w/v alginate solution. However, at 36 hours after printing, PAEC with 0.1 or 1.0 mg/ml nanoparticles were 16% or 35% less viable than cells without nanoparticles, respectively. The viability loss was independent of the printing process. Cell viability continued to decrease with time up to 60 hours after cell printing (ANOVA, p<0.0001). In a long term assay (FIG. 11B), endothelial cell viability similarly decreased nearly 22% with 1.0 mg/ml iron oxide nanoparticles in the alginate 72 hours after printing compared to samples without nanoparticles (ANOVA, p<0.0001). No further cell viability decrease was observed from 72 hours to 144 hours, showing that cells maintained their viability following the initial nanoparticle toxicity effect.

Increased nanoparticle concentration decreased cell viability, but no additional decrease was observed with printing (FIG. 11A). PAEC encapsulated in alginate with 1.0 mg/ml nanoparticles showed 20% lower viability than cells with 0.1 mg/ml nanoparticles and 36% lower than the control, suggesting a nanoparticle concentration dependent effect on cell viability. This decreased viability was observed 36 and 60 hours after printing, but the printing process itself did not affect cell viability.

### Effect of Alginate Concentration on Printed Cell Viability

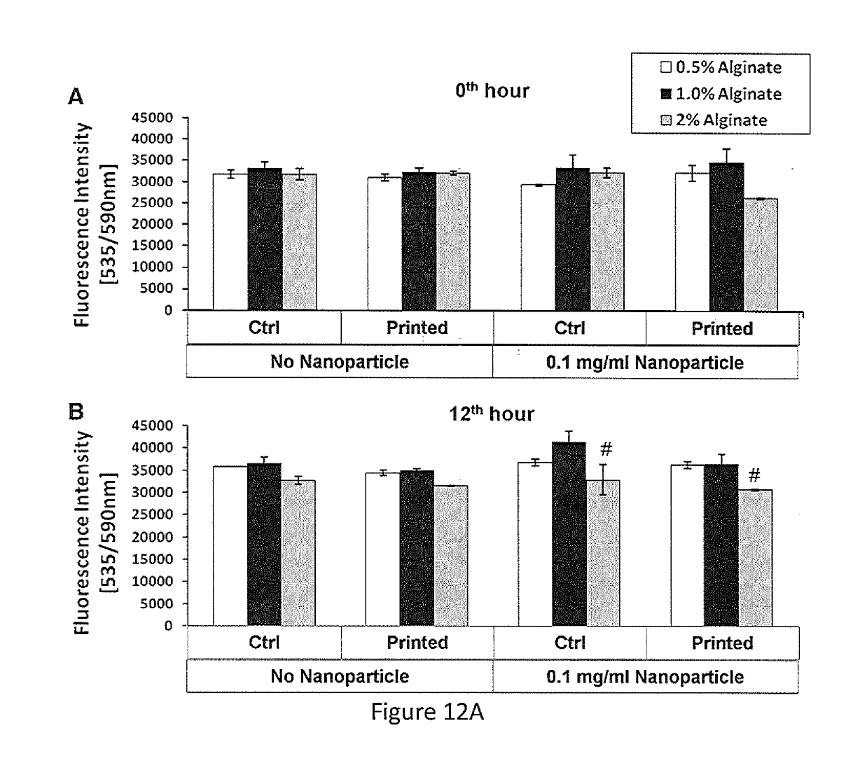

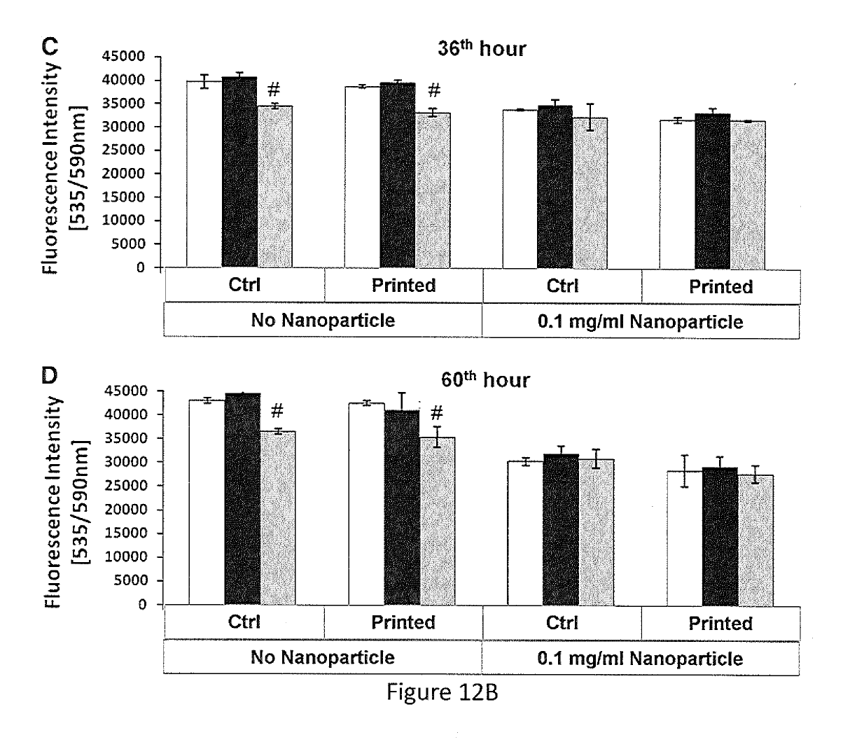

Whether alginate concentration, which effectively alters biopolymer viscosity, affected printed cell viability was evaluated. Immediately following printing, there was a 20% viability decrease for cells printed with nanoparticles in 2% w/v alginate as compared to the 1% w/v alginate (FIG. 12). Twelve hours after printing, lower viability was also observed for control cells with nanoparticles in the 2% w/v alginate. This decreased cell viability for cells with nanoparticles in the 2% w/v alginate solution was no longer observed at later time points, primarily because cell viability decreased in the samples with nanoparticles in 0.5% or 1% alginate. Interestingly, in cell samples without nanoparticles, cell viability decreased for both control and printed cells without nanoparticles in the 2% w/v alginate solution at 36 and 60 hours (FIGS. 12C, D). Overall, cells without nanoparticles in the 0.5% and 1% w/v alginate solutions demonstrated an increase in Alamar blue fluorescence over time, which could represent increased cell number or increased cell metabolism. No cell samples in alginate with nanoparticles, and no cell samples in 2% alginate, showed this increase in viability with time. This effect also was independent of printing.

### Effect of Cellular Nanoparticle Uptake on Printed Cell Viability

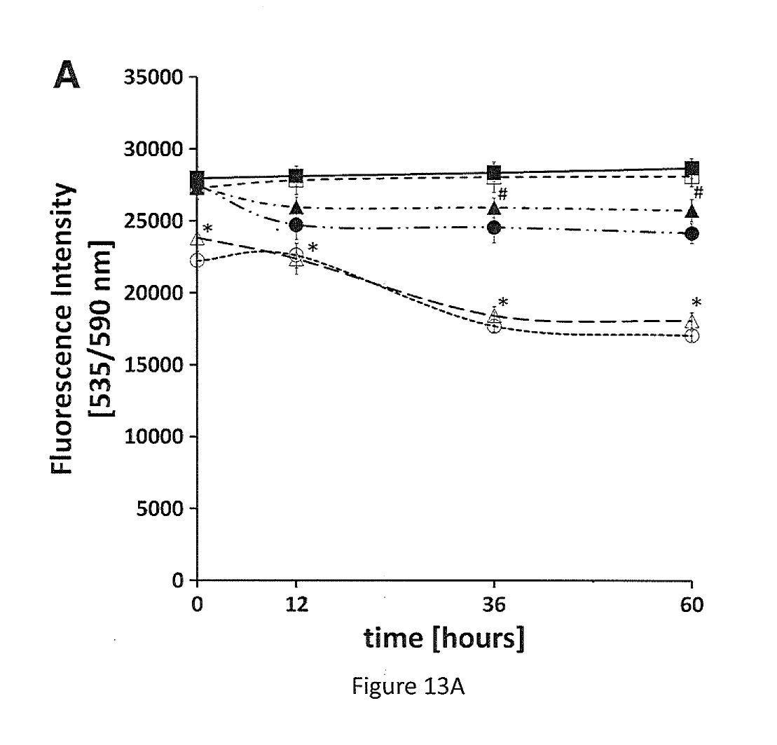

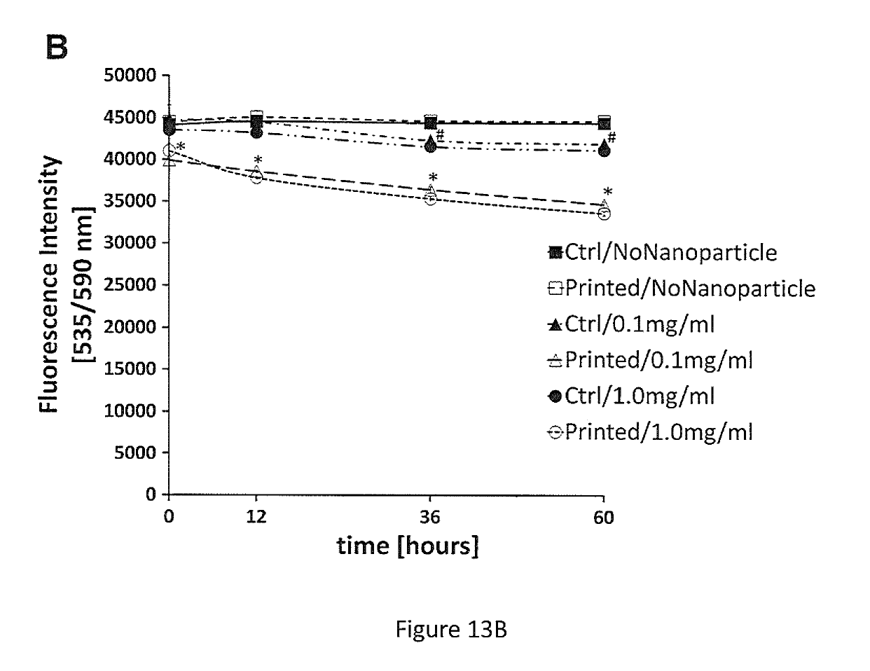

Magnetically labeled cells, internally loaded with iron oxide nanoparticles, can be used to track and move cells printed within a tissue engineered structure. The viability of nanoparticle loaded cells was examined after printing in 1% alginate and an initial dispensing pressure of 5 psi. Both control and printed samples without nanoparticles showed increased viability at timepoints up to 60 hours. However, a steep decrease in cell viability was observed from 0 to 36 hours for both control and printed cells loaded with either 0.1 or 1.0 mg/ml nanoparticles (FIG. 13). Printed cells showed the most dramatic change, with a 40% decrease in the Alamar blue fluorescence when compared to printed cells without nanoparticles at 36 hours. This viability change was in direct contrast to the lack of printing effect for samples with nanoparticles in the alginate. While early cell viability was significantly decreased, there was no significant change after 36 hours, suggesting stabilization of the remaining cell population. When printing pressure was decreased to 2 psi, cell viability increased almost 20% (FIG. 13).

### Nanoparticle Manipulation Inside the Alginate

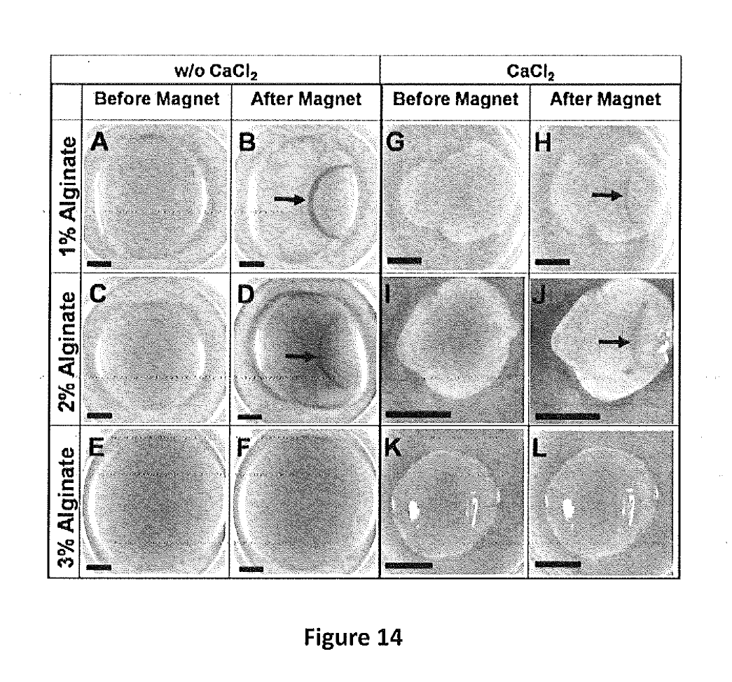

Nanoparticles were magnetically manipulated within the alginate to determine if nanoparticles could be used to move bioactive factors after printing. 1,0 mg/ml nanoparticles were homogenously distributed in 1%, 2%, and 3% w/v alginate, printed in bulk samples, and left as a viscous liquid or crosslinked with calcium chloride to form a gel (FIGS. 14A, C, E; FIGS. 14G, I, K). Nanoparticles printed in either 1% or 2% w/v alginate without calcium chloride moved towards the NdFeB magnet placed under the cell culture dish within a minute (FIGS. 14B, D; arrows indicate nanoparticles at the magnet edge). However, no nanoparticle movement was observed in the 3% w/v alginate solution, likely due to the high alginate solution viscosity (FIG. 14F). When the samples were crosslinked with calcium chloride, nanoparticles similarly moved toward the magnet edge in the 1% and 2% w/v alginate, but not 3% alginate, (FIGS. 14H, J, L). However, the nanoparticles moved more slowly, and less spatial repositioning of nanoparticles was observed.

Movement of a single spherical magnetic nanoparticle at steady state in an external magnetic field is driven by the force due to the magnetic field gradient and opposed by the force due to viscous drag (Holligan et al., 2003, Nanotechnology 14:661; Kalambur et al., 2005, Nanotechnology 16:1221) which is given by:

F_{mag}=(\vec{m}·\vec{∇})\vec{B} (1)

Fᵥᵢₛ=3πηd\vec{v} (2)

(where m, B, n, d, v are the nanoparticle net magnetic moment, magnetic field, suspending medium viscosity, nanoparticle diameter, and instantaneous nanoparticle velocity, respectively. Considering a one-dimensional problem along the centerline of the magnet (x axis) at steady state, a force balance between equations (1) and (2) leads to a velocity given by:

$$\vec{v} = \frac{Ms \cdot {d}^{2}}{18 η} \frac{d B}{d x} \qquad ( 3 )$$

where Ms is the particle saturation magnetization and dB/dx is the magnetic field gradient along the central axis. As seen from equation (3), the nanoparticle velocity inside the alginate is inversely proportional to the medium viscosity and directly proportional to the magnetic field gradient. So in a higher viscosity biopolymer, a stronger magnetic field will be needed to move the same nanoparticle. Even though nanoparticles did not noticeably move in 3% w/v alginate, and nanoparticle movement decreased with crosslinking, it may be possible to move these nanoparticles in the more viscous biopolymer with a stronger magnet.

### Endothelial Cell Movement Inside the Alginate

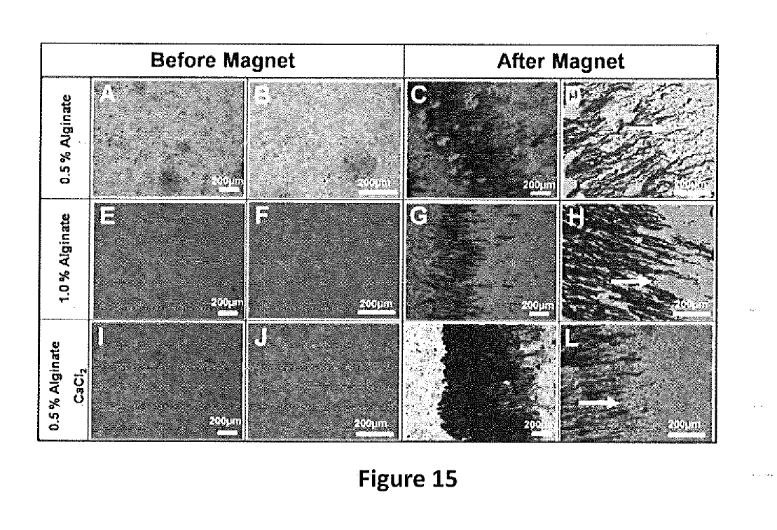

Whether cells loaded with magnetic nanoparticles could be moved within the alginate biopolymer was evaluated. PAEC magnetically labeled with nanoparticles were initially homogenously distributed in 0.5% and 1% w/v alginate (FIGS. 15A, E, I; higher magnification in FIGS. 15B, F, J). Magnetically labeled cells moved toward the NdFeB magnet placed under the cell culture dish (FIGS. 15C, G, K). Images were taken 6 hours after magnet placement. At higher magnification, individual cells were seen at the magnet edge (arrows in FIGS. 15D, H and L). Isolated nanoparticles can also be seen in the alginate, which are likely artifacts of incomplete nanoparticle removal from the cell solution when it was mixed with alginate. Magnetically labeled cells continued to cluster at the magnet edge in the cross-linked alginate, but no movement was observed in alginate concentrations higher than 1%.

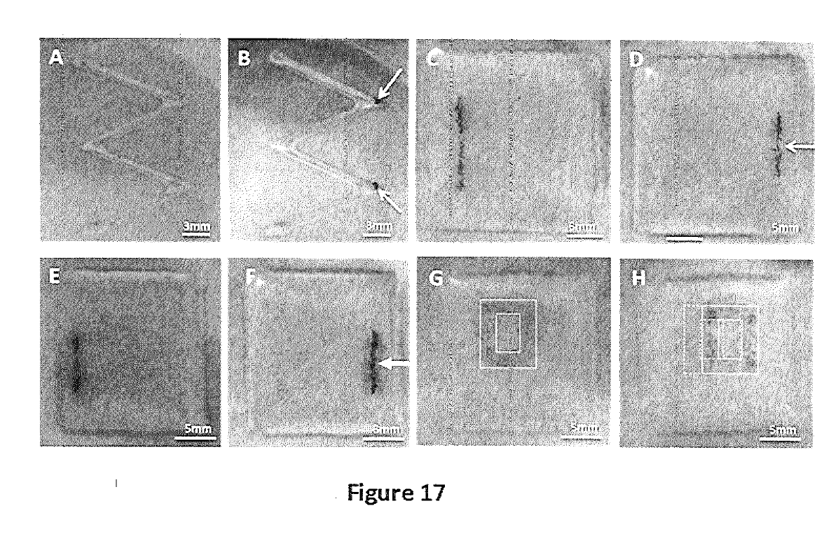

Specified patterns of nanoparticles and magnetically labeled cells were printed and moved using a magnetic field. 1% alginate with iron oxide nanoparticles was printed in a pattern (FIG. 17A), and a magnetic field was used to move the nanoparticles to the printed pattern tips (FIG. 17B). Basic shapes (lines and rectangles) of either nanoparticles (FIGS. 17C, 17D, 17G, 17H) or magnetically labeled cells (FIGS. 17E, 17F) were moved to new locations while maintaining the original pattern.

### MicroCT Scan of 3D Deposited Tissue Scaffold

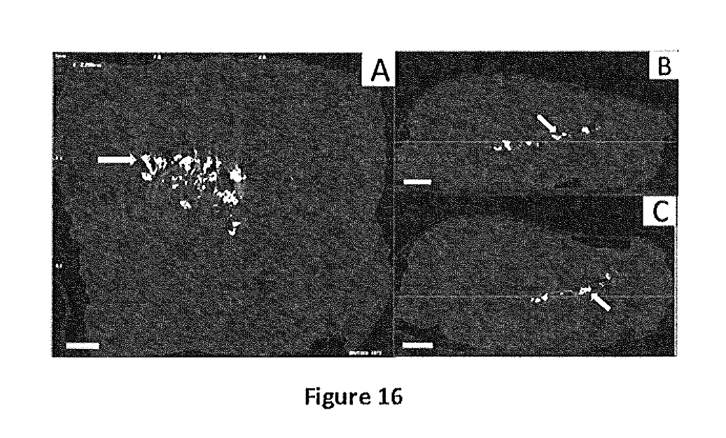

Magnetic nanoparticles printed within three-dimensional alginate scaffolds were imaged by MicroCT to determine if nanoparticle printing would allow non-invasive tracking of bioactive factors and cell location in tissue engineering structures. A nanoparticle-alginate prepolymer solution was encapsulated in alginate biopolymer solution using layer-by-layer deposition with the solid freeform fabrication based direct cell writing system. Printed nanoparticle clusters are clearly visible by MicroCT scan of the three-dimensional tissue scaffold (arrows, FIG. 16),

### Example 2

### Effects of Printing Parameters and Scaffold Properties

The following materials and methods were used in Example 2.

### Scaffold Material

Sodium alginate powder (FMCBioPolymer, Drammen, Norway) was dissolved in deionized water at 1, 2 and 3% w/v concentrations. An ionic cross-linking solution was prepared by dissolving calcium chloride, CaCl2 (BDH Chemicals, Poole, UK), in deionized water. NanoArc magnetic iron oxide nanoparticles (20-40 nm diameter, Alfa Aesar, Ward Hill, Mass.) were used in all experiments. Sodium alginate-magnetic nanoparticle solutions were prepared by vigorously mixing sodium alginate with increasing concentrations of iron oxide nanoparticles to achieve a homogeneous nanoparticle distribution.

### Cell Culture

PAEC were isolated by the collagenase dispersion method and maintained in low glucose Dulbecco's Modified Eagle's Medium (DMEM) supplemented with 5% fetal bovine serum, 1% penicillin-streptomycin and 2% glutamine (Invitrogen, Carlsbad, Calif.). Culture medium was changed every 48 hours, and cells between passages 4 and 9 were used. Prior to printing, cells were gently mixed at a concentration of 1.5×10̂5 cells ml̂-1 in a sodium alginate solution to ensure uniform cell distribution. For magnetically labeled cells, PAEC in 100 mm tissue culture dishes were loaded with different nanoparticle concentrations and incubated at 37° C., 5% CO2 for 24 hours. Cellular nanoparticle uptake was confirmed by transmission electron microscopy, which showed nanoparticle clusters in the cell cytoplasm.

### Cell Dispensing System

A proprietary solid freeform fabrication-based direct cell writing system was developed as described elsewhere herein to create three-dimensional tissue constructs by dispensing cells and biopolymers into predefined patterns. The direct cell writing system used in this study operates at room temperature and low-pressure conditions to facilitate deposition of living cells, growth factors or other bioactive compounds in controlled amounts with precise spatial positioning. Pneumatic microvalves (EFD, East Providence, R.I.) were used to apply printing pressures of 5 and 40 psi. Sodium alginate was chosen as the scaffold biopolymer. Alginate-nanoparticle—cell mixtures with 0, 0.1 or 1.0 mg ml̂-1 nanoparticle concentration were printed with 410 gm and 250 gm diameter nozzles. All samples were dispensed as 0.3 g of bulk material with a sample size of three. After dispensing, each sample was immediately submerged in a 5.0% w/v CaCl2 cross-linking solution for 5 minutes, placed in supplemented medium and returned to the incubator.

### Alamar Blue Cell Viability Assay

Alamar blue quantitatively measures cell metabolic activity using an oxidation—reduction indicator that fluoresces and changes color in metabolically active cells. Cross-linked alginate-cell solutions in six well plates were incubated with 2 ml supplemented medium and 200 1.1,1 Alamar blue solution (AbD Serotec Ltd, Oxford, UK). After 4 hours of incubation at 37° C. in 5% CO2, 100 gl of medium from each well was transferred into a 96-well flat-bottomed black assay plate, and fluorescence was measured at 535/590 nm in a GENios microplate reader. 4×10̂4 cells were calibrated to a fluorescence intensity reading of 40,000.

### Nanoparticle Movement in the Scaffold

1.0 mg ml̂-1 magnetic nanoparticles in 1% or 2% w/v alginate were printed using the direct cell writing system at a fixed location (x=2 mm) from a 1 inch diameter NdFeB magnet (K&J Magnetics, Jamison, Pa.) (FIG. 1(a)). Nanoparticle displacement along the magnet center line was imaged at 100 frames s-1 using a Nikon TS100 microscope. Nanoparticle velocity was calculated from the derivative of the transient displacement data.

Experimental nanoparticle velocity observations were compared to theoretical calculations. The net force induced on a superparamagnetic nanoparticle in a viscous medium by an externally applied magnetic field gradient is a balance of the magnetic force (Fmag) and the viscous drag (Fvisc) (Zborowski et al., 1996, ASAIO J. 42:M666-671; Kalambur et al., 2005, Nanotechnology 16:1221-1233):

\vec{F}_{mag}=(\vec{m}·\vec{∇})\vec{B} (1)

\vec{F}_{visc}=3πηd\vec{v} (2)

where m→ is the total nanoparticle magnetic moment, which depends on the nanoparticle material and volume; B→ is the magnetic field; n is the suspending fluid viscosity; d is the nanoparticle diameter and v→ is the instantaneous nanoparticle velocity. For a one-dimensional problem along the magnet centerline (x axis), the nanoparticle velocity v at a steady state can be obtained by balancing the forces from equations (1) and (2) as

$$\vec{v} = \frac{{M}_{s} {d}^{2}}{18 η} \frac{d B}{d x} \qquad ( 3 )$$

where Ms is the particle saturation magnetization and dB/dx is the magnetic field gradient along the center axis. The field intensity was calculated along the center axis (x) of the cylindrical magnet using the following analytical expression (Hatch and Stelter, 2001, J. Magn. Magn. Mater 225:262-276):

$$B ( x ) = \frac{{B}_{r}}{2} [ \frac{x + 1}{\sqrt{{( x + 1 )}^{2} + {r}^{2}}} - \frac{x}{\sqrt{{x}^{2} + {r}^{2}}} ] \qquad ( 4 )$$

where B is the flux density at a point x away from the pole face and parallel to the magnet axis, 1 is the magnet length and r is the magnet radius. Note that the flux direction is normal to the pole surface along the axis. The residual induction of the permanent magnet is Br and is a characteristic of the magnet material. The magnetic flux density derivative, dB/dx, was calculated as

$$\frac{d B}{d x} = \frac{{B}_{r}}{2} [ \begin{matrix}{\{ {( x + 1 )}^{2} + {r}^{2} \}}^{- 1 / 2} - \{ {{( x + 1 )}^{2} [ {( x + 1 )}^{2} + {r}^{2} ]}^{- 3 / 2} \} - \\ {( {x}^{2} + {r}^{2} )}^{- 1 / 2} + {{x}^{2} ( {x}^{2} + {r}^{2} )}^{- 3 / 2}\end{matrix} ] . \qquad ( 5 )$$

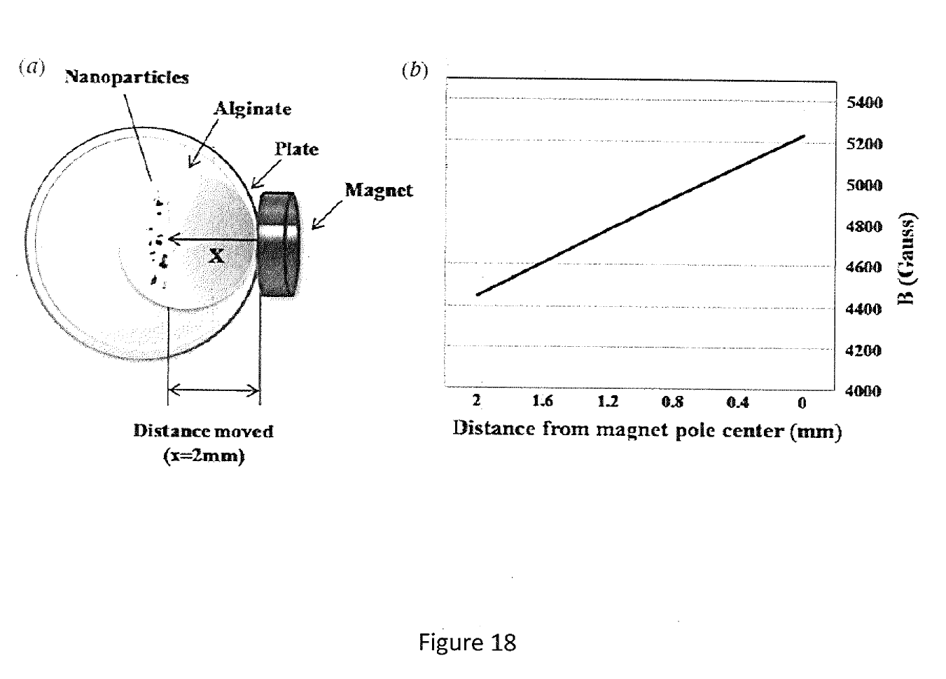

Theoretical calculations were compared with experimental results by substituting appropriate materials properties for the magnet and nanoparticles used. The NdFeB had magnetic flux density Br=14 800 G, radius r=25 4 mm and length l=12.7 mm. The magnetic field along the center axis of the magnet reached a maximum of 5200 G near the pole and decreased to 4400 G 2 mm away from the magnet (FIG. 18b). Saturation magnetization Ms of the iron oxide nanoparticles was taken from the literature, where it was measured to 66 emu g-1 using a SQUID magnetometer (Jain et al., 2005, Mol. Pharmacol 2:194-205).

### Viscosity Measurement

Viscosity of 1, 2 and 3% alginate was measured using a rotating viscometer (Brookfield Co. HBTD, Stoughton, Mass.) at 10, 20 and 50 rpm. 1 mg ml̂-1 and 5 mg ml̂-1 iron oxide nanoparticles were mixed with alginate and viscosity was measured.

### Statistical Analysis

Samples were statistically compared using Student's t-test. Statistical significance was established at either p<0.05 (#) or p<0.01 (*). Two-way ANOVA was used to compare changes over time, with statistical significance established at p<0.0001. The results of this Example are now described.

The following results are provided for Example 2.

### Effect of Nozzle Size and Printing Pressure on Cell Viability

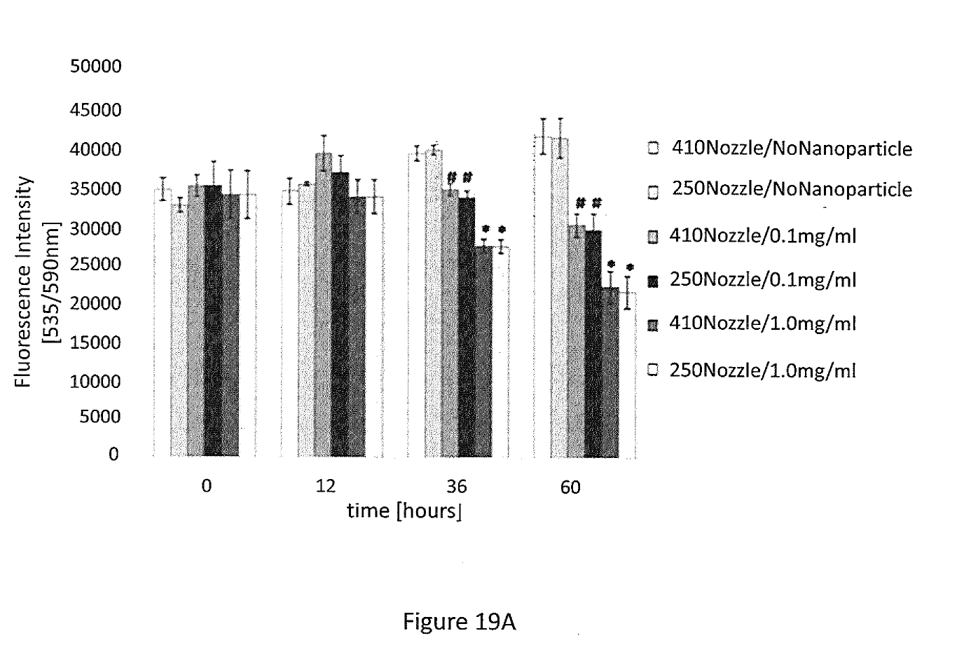

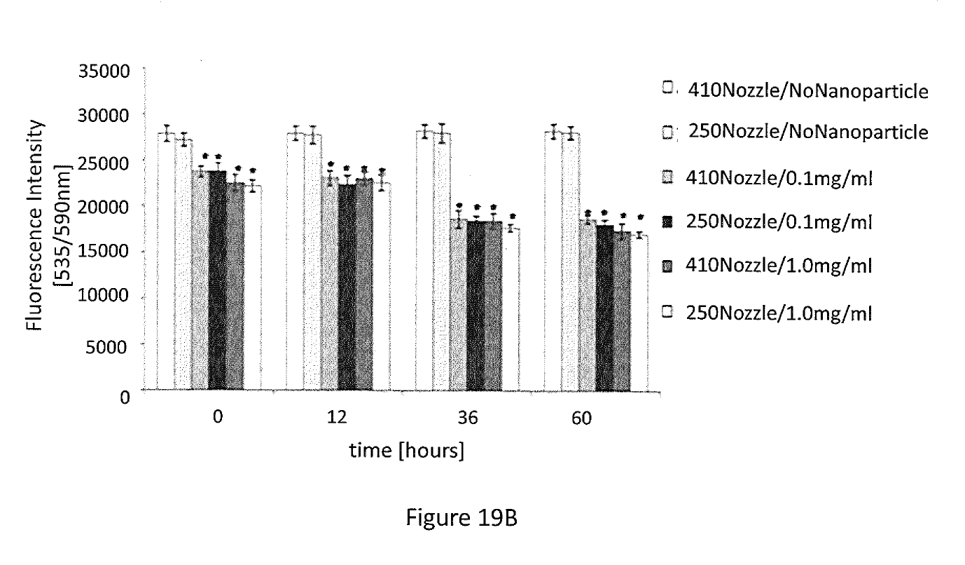

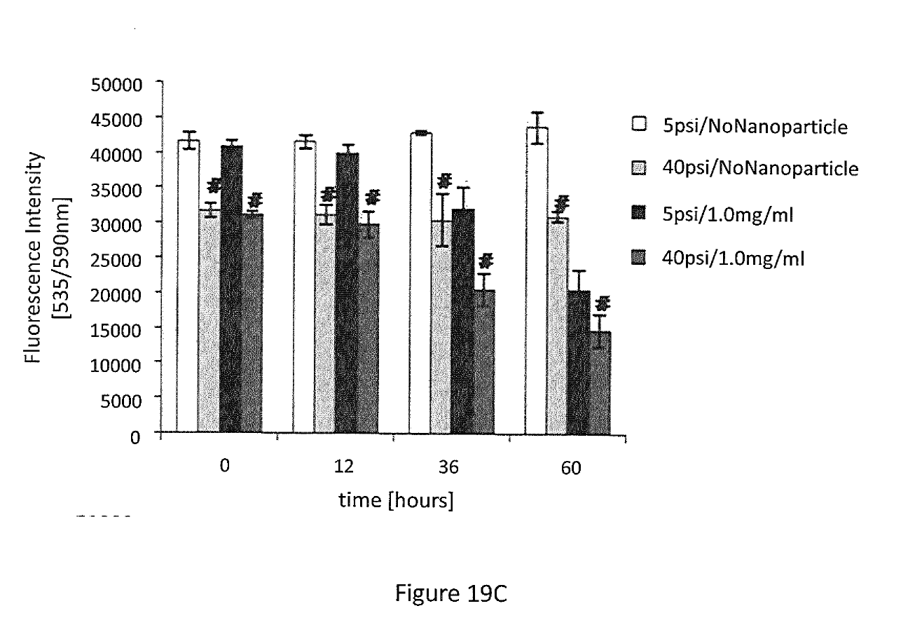

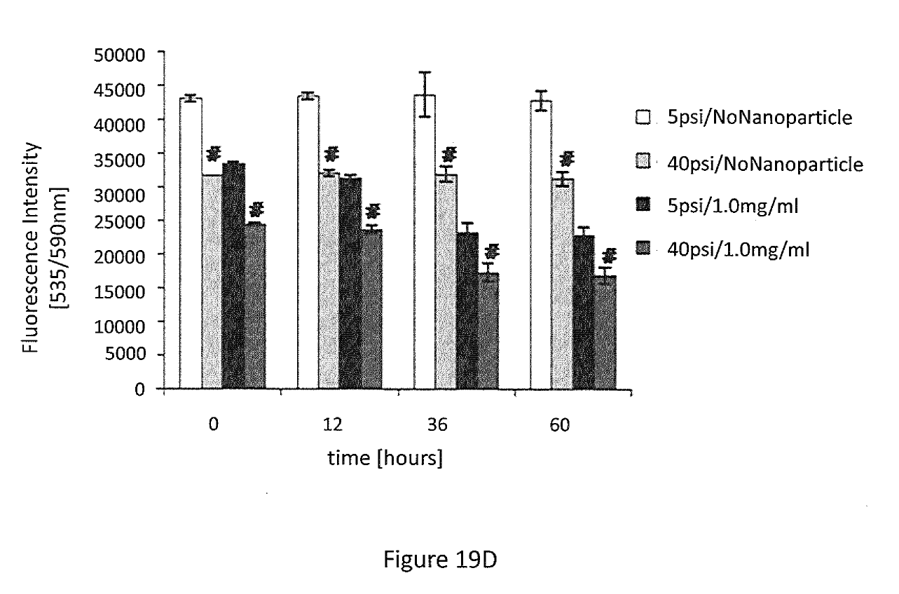

Bioprinting magnetic nanoparticles in a biopolymer scaffold may provide an effective means to track and manipulate bioactive factors in tissue engineered structures. It is shown herein that while nanoparticles in the alginate slightly decreased endothelial cell viability, nozzle size had no significant effect (FIGS. 19a). At 0 and 12 hours after printing, cell viability did not change significantly for printed cells with 0, 0.1 and 1.0 mg ml̂-1 nanoparticles in a 1% alginate solution. However, 36 hours after printing, PAEC with 0.1 or 1.0 mg ml̂-1 nanoparticles were 16% or 35% less viable than cells printed without nanoparticles, respectively. The viability loss was independent of nozzle size. Cell viability continued to decrease up to 60 hours after cell printing (ANOVA, p<0.0001); however, long-term experiments showed no further cell viability decrease after 60 hours (data not shown).

Magnetically labeled cells, internally loaded with iron oxide nanoparticles, could be used to image and move cells printed within a tissue engineered structure. Nanoparticle loaded cell viability was examined after printing. While viability was unchanged for printed cells without nanoparticles, viability decreased from 0 to 36 hours for printed cells loaded with either 0.1 or 1.0 mg ml̂-1 nanoparticles (FIG. 19b). However, nozzle size did not affect cell viability. Nanoparticle loaded cells printed with either a 250 and 410 gm diameter nozzle demonstrated 36% viability loss compared to cells printed without nanoparticles at 36 hours. While early-cell viability was decreased, there was no significant change after 36 hours, suggesting stabilization of the remaining cell population.

Increasing printing pressure from 5 psi to 40 psi decreased cell viability by 25% when nanoparticles were in the alginate (FIGS. 19c), and 26% for magnetically labeled cells (FIG. 19d) immediately following bioprinting. Cell viability continued to decrease in a similar manner for both nanoparticle conditions and printing pressures. The combined effect of printing pressure and nanoparticles affected cell viability in an additive manner and at different times, suggesting no interaction between the two printing parameters.

### Effect of Nanoparticles on Alginate Viscosity

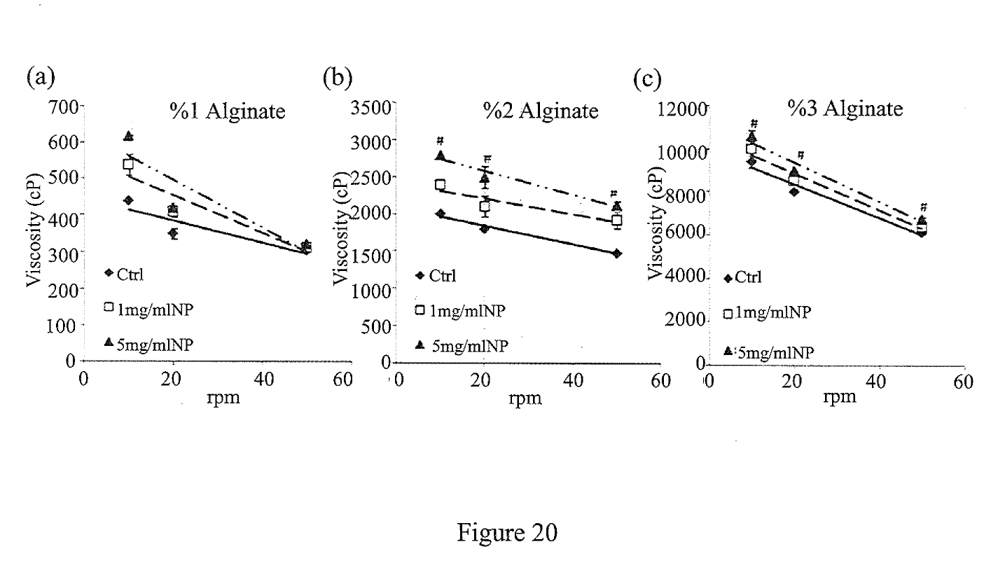

Biopolymer scaffold viscosity affects printing resolution; therefore, alginate viscosity at different concentrations and with nanoparticles was measured. Alginate viscosity increased with alginate percentage and decreased with rotational velocity. At 20 rpm, viscosity increased from 400 cP for 1% alginate to 1250 cP for 2% alginate and 8000 cP for 3% alginate (FIGS. 20a-20c). Alginate viscosity decreased more than 25% with increasing velocity (strain rate) for all concentrations, with 3% alginate showing the most dramatic non-Newtonian properties. 1.0 mg ml̂-1 and 5.0 mg ml̂-1 iron oxide nanoparticles did not significantly affect 1% alginate viscosity, at least within the measurement capability of the system (FIG. 20a). However, in 2% (FIGS. 20b) and 3% (FIG. 20c) alginate, 5.0 mg ml̂-1 nanoparticles resulted in a statistically significant increase in alginate viscosity (p<0.05).

### Effect of Alginate Viscosity and Nanoparticle Cluster Size on Nanoparticle Velocity

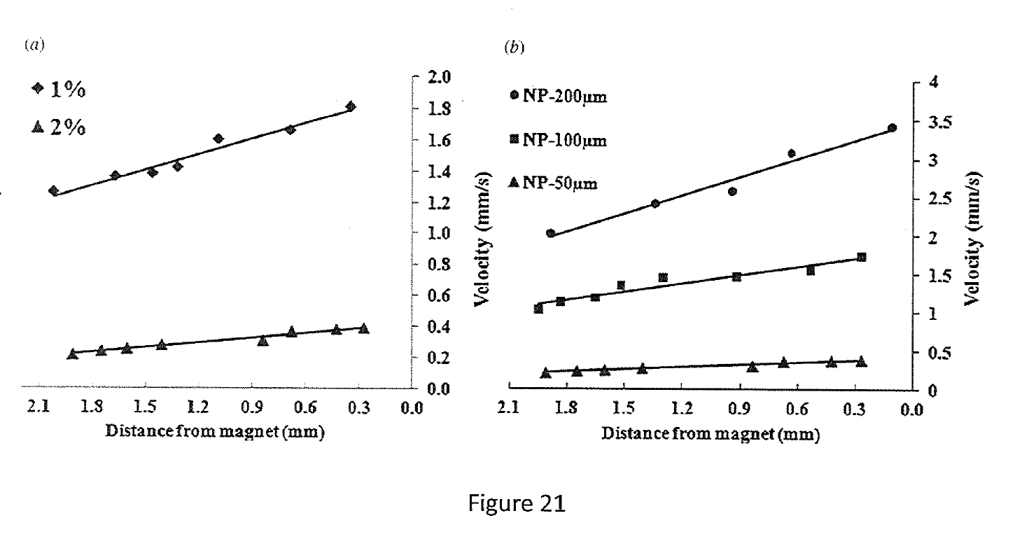

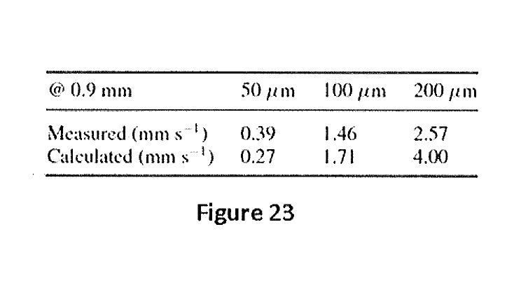

Nanoparticle velocity in the alginate biopolymer was quantified as a function of alginate viscosity and nanoparticle cluster size. Nanoparticle velocity was four times faster in 1% alginate than 2% alginate (FIG. 21a). Due to limited testing length, the nanoparticles did not reach a constant velocity. Instead, they accelerated at 0.385 mm s-2 in 1% alginate, and much slower at 0.088 mm s-2 in 2% alginate. While ideally nanoparticles would be mono-dispersed in the alginate biopolymer, particles aggregated in clusters, particularly when a magnetic field was applied. Velocities for three nanoparticle cluster sizes were compared in 2% alginate. The larger cluster sizes moved faster in the alginate, which agreed with the theoretical calculation that nanoparticle velocity increases with the square of particle diameter. In our experiments, 200 gm sized clusters moved five times faster than 50 gm sized clusters when the nanoparticles were 0 9 mm from the magnet (FIG. 21b). Experimentally determined nanoparticle cluster velocities showed good agreement with calculated velocities (FIG. 23).

### Effect of Nanoparticles on Printing Resolution

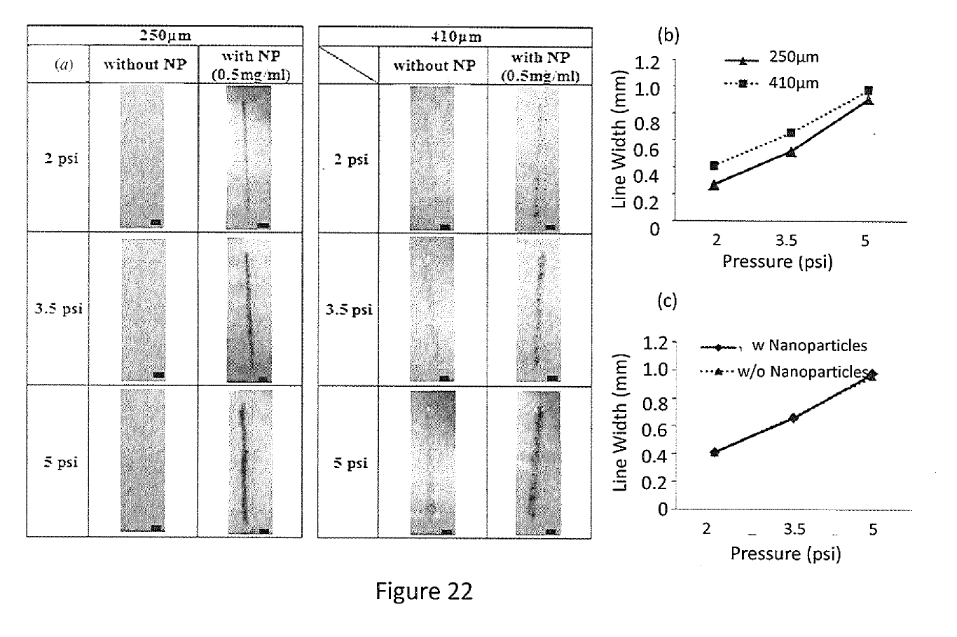

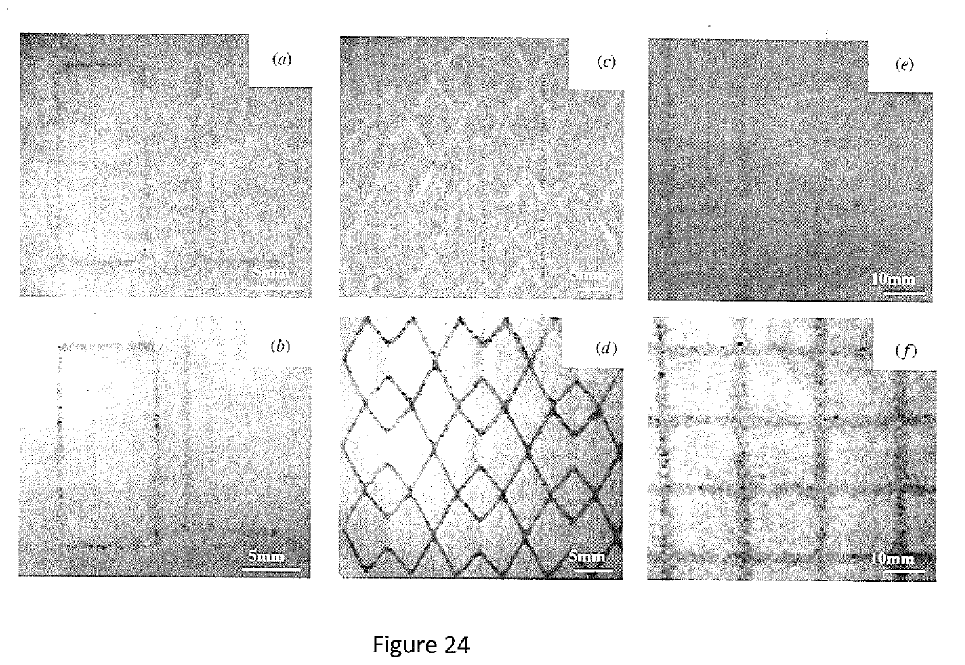

Nanoparticles may alter biopolymer flow rate, and therefore affect bioprinting resolution. Lines were printed with and without nanoparticles with 250 μm and 410 μm nozzles at 2, 3.5, and 5 psi printing pressure (FIG. 22a). Printed lines were of the same width as the nozzle diameter at the low 2 psi pressure. As printing pressure increased to 3.5 and 5 psi, the printed line width increased linearly to more than twice the nozzle diameter (FIG. 22b). However, the presence of nanoparticles in alginate did not change the printed line width (FIG. 22c). Printing resolution was maintained with nanoparticles, as shown by the complex patterns printed with (FIGS. 24b, 24d, 24f) and without (FIG. 24a, 24c, 24e) nanoparticles.

### Example 3

### Nanoparticle Size and Composition Studies

Bare iron oxide vs. polymer-coated nanoparticles may create different cell responses to bioprinting. While studies of nanoparticle size and composition effects on cells have been performed, none have examined cell-nanoparticle interactions in a mechanical system such as the bioprinting nozzle of the present invention.

As endothelial cells are critical to vasculature formation in tissue engineering structures, porcine aortic endothelial cells (PAEC) can be used as described herein. However, other cell types, such as hepatocytes and fibroblasts, can be studied to determine if cell-nanoparticle interactions are cell type specific. Both 5 and 30 nM nanoparticles (NN labs) may be used as described herein. The critical outcome for each study is cell viability and function. Endothelial cell function can be assessed through nitric oxide synthase (eNOS), which allows cells to produce nitric oxide (NO), a critical factor in vascular homeostasis.

### Nanoparticle Size

Small (5 nm) or medium (30 nm) nanoparticles at 0, 0.1, or 0.5 mg/ml can be mixed with 1% w/v sodium alginate biopolymer or incubated with cells for 24 hours. Further, 5×105 cell/ml can be mixed with the alginate-nanoparticle solution. Next, 0.3 g samples can be bioprinted using 250 μm nozzles, cross-linked in 5.0% CaCl₂, placed in supplemented medium, and returned to the incubator.

Cell viability in 3D tissue constructs can be assessed using Alamar blue, which measures cell metabolic activity. Up to 72 hours after printing, cross-linked alginate-nanoparticle-cell samples can be incubated with Alamar blue (AbD Serotec) for 4 hours, and fluorescence can be measured at 535/590 nm in a microplate reader. Cell viability can be confirmed using a Live/Dead assay (Invitrogen) as per manufacturer instructions. Live or dead cell number can be counted in printed alginate samples using confocal fluorescent microscopy (Olympus IX81). The cell death mechanism, whether by apoptosis or necrosis, can be measured via annexin V-propidium iodide labeling. Bioprinted nanoparticle-alginate-cell samples can be labeled with annexin V-fluorescein and propidium iodide as per manufacturer instructions (BD Pharmingen) and analyzed immediately by confocal fluorescent microscopy.

Nanoparticle biochemical and mechanical effects on cells, specifically ROS and actin cytoskeleton disruption, as well as cell function, can be assessed in printed samples. Cells printed with different nanoparticle sizes can be labeled for ROS using the Live Green Reactive Oxygen Species Detection Kit (Invitrogen) according to manufacturer instructions and imaged in a confocal microscope. Actin and eNOS in printed PAEC can be imaged by confocal microscopy. Nanoparticle-alginate-cell samples can be fixed in 4% paraformaldehyde, permeabilized with 0.1% Triton X-100 in PBS, and labeled for actin (rhodamine phalloidin, Invitrogen, 1 unit/well) or an anti-eNOS antibody (BD Biosciences) and nuclei (bisbenzimide, Sigma, 1 μg/mL).

### Nanoparticle Composition.

The effect of nanoparticle composition on nano-bioprinted cell viability can be assessed by coating nanoparticles with polyethylene glycol (PEG). PEG has low toxicity and is commonly used in biomedical applications. 10 mg dried iron oxide nanoparticles can be dispersed 3 mM methoxy-PEG-silane (Shearwater Polymers). The mixture can be sonicated and incubated at 60° C. for 4 h. Nanoparticles can be washed with toluene and ethanol. The nanoparticle coating can be characterized both before and after incubation in medium for 24 to 72 hours. Coated nanoparticle size and coating thickness can be measured by TEM. Cell viability, death mechanism, function, and nanoparticle biochemical and mechanical effects on cells can be measured as described.

From these experiments, it is expected that smaller nanoparticles may have less damaging cell interactions during the printing process, yet they can be taken up more readily by cells. It is anticipated that PEG coating may diminish nanoparticle effects on cell function through decreased intracellular ROS formation. A PEG coating can be selected due to its wide use in nanoparticle drug delivery, the capability of further modifying PEGylated nanoparticles, and the relatively simple coating process. In the alternative to PEG coating, nanoparticles can be coated with the polysaccharide dextran.

### Example 4

### Nanoparticle Location Relative to Cell

Bioprinted nanoparticle cell effects appear directly related to whether nanoparticles are outside or inside cells, and that nanomanufacturing process parameters such as printing pressure have different cellular effects depending on nanoparticle location.

Nanoparticle size and concentration can be selected based on the results of Example 3, above. Printing pressure (5 psi), nozzle size (250 μm), and scaffold material (1% alginate) can be held constant. Nanoparticle location and uptake efficiency can be visualized by TEM. Samples can be fixed with 4% paraformaldyde and 2% osmium tetroxide, dehydrated in graded ethanol, and embedded in PolyBed 812 (Polysciences). Samples can then be sectioned en face, stained with uranyl acetate and bismuth subnitrite, and examined with a JEOL 1010 TEM.

### Nanoparticles in Scaffold, Cells, or on Cell Membranes

For nanoparticles in the scaffold, PEG-coated nanoparticles can be mixed in the alginate biopolymer. For nanoparticles inside cells, PAEC can be incubated with PEG-coated nanoparticles for 24 hours. For nanoparticles on the cell membrane, PEG-coated nanoparticles can be labeled with antibodies to vascular cell adhesion molecule (VCAM, Research Diagnostics) by biotinylation. Methoxy-PEG-silane can be added to N-hydroxysuccinimide-biotin (Sigma) and triethylamine in dichloromethane and acetonitrile overnight. Nanoparticles coated with biotinylated PEG can be incubated with neutravidin followed by the biotinylated VCAM antibody. VCAM functionalized nanoparticles can be incubated with PAEC for 24 hours. For each configuration, nanoparticle location relative to cells can be confirmed by TEM. 5×105 PAEC/ml can be added to alginate immediately prior to printing. Printed samples can be assessed for cell viability, function, and nanoparticle biochemical and mechanical effects as described.

### Cell Magnetic Labeling Efficiency

Maintenance of cell viability and function can be balanced with cell magnetic labeling by finding the minimum nanoparticle loading concentration required for cells to move at 100 μm/sec and achieve a 2 fold increase in μCT signal intensity. Cells and nanoparticles can be printed using the direct cell writing system at a fixed location from an NdFeB magnet. Nanoparticle displacement can be imaged at 100 frames/second using a Nikon TS 100 microscope, and nanoparticle velocity calculated as the derivative of the transient displacement data. Experimental nanoparticle velocity observations can be compared to theoretical calculations. The net force on a magnetic particle in a viscous medium by an externally applied magnetic field is a balance of the magnetic force (Fmag) and the viscous drag (Fvisc) according to equations (1) and (2), above, where m→ is the total nanoparticle magnetic moment, which depends on the nanoparticle material and volume; B→ is the magnetic field; n is the suspending fluid viscosity; d is the nanoparticle diameter and v→ is the instantaneous nanoparticle velocity. For a 1D problem along the magnet centerline (x axis), the nanoparticle velocity at steady state is obtained by balancing magnetic and viscous drag forces. Cell saturation magnetization can be calculated from experimental data and compared with measured nanoparticle Ms (66 emu/g) to determine magnetic labeling efficiency [35]. Samples with a defined internal region of magnetically labeled cells can be imaged using a μCT scanner (SkyScan 1172).

It is expected that cell effects associated with nano-bioprinting magnetically labeled cells can be decreased by cell nanoparticle uptake after printing, lower concentrations of PEG-coated nanoparticles, and nanoparticles attached to the cell surface. While each method has its advantages and disadvantages, it can be determined which method best balances cell function with labeling efficiency. This method can improve the potential of magnetically functionalized tissue engineering scaffolds for moving and tracking cells. If cells do not take up PEGylated particles, dextran can be used. If endothelial cells do not express adequate VCAM to be magnetically labeled using these antibodies, selectins can also be used.

While the invention has been disclosed with reference to specific embodiments, it is apparent that other embodiments and variations of the invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

What is claimed is:

1. A process for manufacturing complex structure comprising:
designing a printable structure via a computer-operable software application;
converting the designed structure into a heterogeneous material and multi-part assembly model; and
printing the designed structure using a device comprising a plurality of differentiated, specialized nozzles, wherein at least one of the nozzles is specialized for the deposition of at least one material comprising a magnetic particle.

2. The process of claim 1, wherein the structure is a tissue scaffold.

3. The process of claim 1, wherein the material comprises a cell.

4. The process of claim 1, wherein the material comprises a magnetically labeled bioactive factor.

5. The process of claim 4, further comprising repositioning the magnetically labeled bioactive factor on or within the structure after initial deposit via a magnetic field after printing the magnetically labeled bioactive factor.

6. The process of claim 1, further comprising repositioning the material comprising a magnetic particle via a magnetic field after printing at least the at least one material comprising a magnetic particle.

7. The process of claim 1, wherein the magnetic particle is a superparamagnetic nanoparticle.

8. The process of claim 7, wherein the superparamagnetic nanoparticle has a diameter between about 5-30 nm.

9. The process of claim 7, wherein the superparamagnetic nanoparticle comprises iron oxide.

10. The process of claim 1, further comprising using Boolean, scaling, smoothing, or mirroring to modify the design prior to conversion into a heterogeneous material and multi-part assembly model.

11. The process of claim 1, wherein the designing further comprises incorporating data taken from MRI, CT or other patient specific data into the designed structure.

12. The process of claim 1, wherein the designing further comprises incorporating a biomimetic and non-biomimetic feature into the designed structure.

13. The process of claim 2, wherein printing the material comprising a magnetic particle comprises depositing magnetically labeled cells or magnetically labeled biological factors.

14. The process of claim 13, further comprising improving histological accuracy, cell ratios, and spatial patterning of cells in the printed structure.

15. The process of claim 1, wherein the structure comprises an artificial organ, an artificial vasculature or channel system, or a sample for cytotoxicity testing.

16. The process of claim 1, wherein the structure comprises a biochip, biosensor, bionic, cybernetic, mechanoactive, or a bioactive tissue scaffold.

17. The process of claim 1, wherein the structure is used in drug delivery.

18. A multi-nozzle biopolymer deposition apparatus comprising:
a data processing system which processes a designed scaffold model and converts it into a layered process tool path;
a motion control system driven by the layered process tool path; and
a material delivery system comprising a plurality of differentiated, specialized nozzles for simultaneously depositing a plurality of biopolymers having different viscosities, thereby constructing a scaffold from the designed scaffold model, wherein at least one of the nozzles deposits at least one magnetically labeled material,

19. The apparatus of claim 18, wherein the at least one magnetically labeled material is a magnetically labeled bioactive factor.

20. The apparatus of claim 18, wherein the at least one magnetically labeled material is a cell.

21. The apparatus of claim 18, wherein the data processing system utilizes Boolean, scaling, smoothing, or mirroring to modify the designed scaffold model.

22. The apparatus of claim 18, wherein the data processing system incorporates data taken from MRI, CT or other patient specific data into the designed scaffold model.

23. The apparatus of claim 18, wherein the data processing system incorporates a biomimetic and non-biomimetic feature into the designed scaffold model.

24. The apparatus of claim 18, wherein the scaffold comprises an artificial organ, an artificial vasculature or channel system, or a sample for cytotoxicity testing.

25. A system for generating a biocompatible structure comprising:
designing a printable structure via a computer-operable software application;
converting the designed structure into a heterogeneous material and multi-part assembly model;
printing the designed structure using a device comprising a plurality of differentiated, specialized nozzles, wherein at least one of the nozzles is specialized for the deposition of at least one material comprising a magnetic particle; and
applying a magnetic field to reposition the at least one material comprising the magnetic particle after its deposition.

26. The system of claim 25, wherein the at least one material comprising the magnetic particle is repositioned prior to completion of all materials being deposited.

27. The system of claim 25, wherein the at least one material comprising the magnetic particle is repositioned after all materials have been deposited.
